(19) United States
(12) Patent Application Publication
(10) Pub. No.: US 2006/0263796 A1
(43) Pub. Date: Nov. 23, 2006

(54) Dyslipoproteinemia associated with venous thrombosis

(76) Inventors: John H. Griffin, Del Mar, CA (US); Hiroshi Deguchi, San Diego, CA (US); Darlene J. Elias, San Diego, CA (US); Natalie Pecheniuk, San Diego, CA (US)

Correspondence Address: MORGAN & FINNEGAN, L.L.P., 3 World Financial Center, New York, NY, 10281-2101, US

(21) Appl. No.: 11/347,632

(22) Filed: Feb. 3, 2006

Related U.S. Application Data
(60) Provisional application No. 60/649,998, filed on Feb. 4, 2005.
(60) Provisional application No. 60/702,890, filed on Jul. 26, 2005.

(51) Int. Cl. C12Q 1/68; G01N 33/92

(52) U.S. Cl. 435/6; 436/71

(57) ABSTRACT

The present invention provides methods of determining a human subject's risk for venous thrombosis based on the finding that venous thrombosis patients have significantly lower levels of large HDL particles, HDL-cholesterol and apolipoprotein Al and higher levels of small LDL particles, LDL-cholesterol and apolipoprotein B. Genotyping showed that venous thrombosis patients differed significantly from controls in CETP genotype and that the CETP genotypes found in subjects with VTE are linked to elevated CETP mass and activity. Methods for determining the level of lipids or lipoproteins in plasma or serum samples to determine risk for venous thrombosis are provided. Methods for reducing the risk of venous thrombosis are also provided.

This application claims the benefit of priority of U.S. Provisional Application Ser. Nos. 60/649,998 filed Feb. 4, 2005 and 60/702,890 filed Jul. 26, 2005, the contents of each of which are incorporated by reference in their entirety.

This invention was made with the assistance of funds provided by the Government of the United States. The government may own certain rights in the present invention, pursuant to grants from the National Institutes of Health, grant numbers R37HL52246, R01HL21544, and NIH-GCRC M0100833.

### FIELD OF THE INVENTION

The present invention relates to methods for diagnosing and treating individuals at risk for venous thrombosis. The present invention further relates to assays for evaluating lipid or lipoprotein concentrations in blood plasma. The present invention also relates to methods of using lipid-lowering drugs to reduce the risk of venous thrombosis.

### BACKGROUND OF THE INVENTION

Venous thromboembolic disease (VTE), a polygenic disease with pathogenic contributions from both genetic and environmental risk factors (1,2), contributes significantly to morbidity and mortality. Various molecular dysfunctions in the protein C pathway, including factor V Leiden (3,4), comprise the currently most common identifiable genetic risk factors for VTE (5). Although dyslipoproteinemia is associated with arterial thrombosis, little is known about the relationships between VTE and plasma lipoproteins. Remarkably, spontaneous VTE is associated with clinically silent atherosclerotic vascular disease (6), and the use of statins reduces VTE (7-9), suggesting a relationship between VTE and dyslipidemia. In fact, subnormal plasma levels of glucosylceramide are found in VTE patients (10). Both glucosylceramide and high density lipoprotein (HDL) enhance the anticoagulant cofactor of activated protein C (10-12), and we speculated that HDL may help protect against VTE (13).

Although many genetic and acquired factors are appreciated as risk factors for venous thromboembolic disease (VTE) (38,39), only recently has evidence emerged for male gender (40,41) and dyslipoproteinemia in males (42) as additional significant risk factors. Surprisingly, silent atherosclerotic vascular disease was associated with venous thrombosis although no causal or mechanistic relationships were suggested (43). In contrast, for many years the association of arterial thrombosis with dyslipoproteinemia, has been well appreciated and multiple mechanisms and successful therapies provide plausible explanations for this association (44-46).

### SUMMARY OF THE INVENTION

The present invention relates to the discovery of novel risk factors associated with venous thrombosis.

In one aspect, the present invention provides a method of determining an individual at risk for venous thrombosis by determination of a deficiency of HDL-cholesterol (HDL-C) or deficiency of large HDL particles (HDL2), apolipoprotein Al (apoAl), and/or apolipoprotein ClII (apoClII) that is not associated with apolipoprotein B (ApoClII-Lp-nonB), where the deficiency is indicative of a risk factor for venous thrombosis.

In another aspect, the present invention provides a method of determining an individual at risk for venous thrombosis by determination of an increase in low density lipoprotein (LDL)-cholesterol (LDL-C) or increase in LDL particles including intermediate density lipoprotein (IDL) particles and/or small LDL particles, where the increase is indicative of a risk factor for venous thrombosis.

The present invention further provides a method of determining an individual at risk for venous thrombosis by determination of an increase of the ratio of apolipoprotein B (apoB) to apolipoprotein Al and/or of an increase of the ratio of LDL-C to HDL-C, where the increase is indicative of a risk factor for venous thrombosis.

In another aspect, the present invention provides a method for determining an individual at risk for venous thrombosis by determination of the cholesterol ester transfer protein (CETP) genotype. This genotype includes the B1/B2 single nucleotide polymorphism of the CETP TaqI polymorphism, where a lower than normal frequency of the B2 allele or a higher than normal frequency of the B1 allele is indicative of a risk factor for venous thrombosis for the individual. It is well known that the presence of the B1 allele of the CETP gene is associated with higher levels of CETP mass and activity, indicating that venous thrombosis is associated with higher levels of CETP mass or activity. Furthermore, the presence of two CETP-linked gene variations, Pro373 and Gln451, that cause low plasma levels of HDL and of large HDL and that are associated with higher than normal levels of CETP mass and activity, are indicative of a risk factor for venous thrombosis.

The invention further provides methods of determining a lipid or lipoprotein concentration in a biological specimen.

The invention further provides methods of administering lipid-lowering drugs or drugs that favorably alter lipoprotein or lipid levels, including drugs that elevate HDL, to inhibit or reduce the risk of venous thrombosis.

These and other aspects of the present invention will be better understood upon a reading of the following detailed description when considered in connection with the accompanying figures.

### BRIEF DESCRIPTION OF THE DRAWINGS

FIG. 1. HDL-associated lipid parameters in VTE and controls by gender. Solid thick lines indicate mean values and the dotted lines indicate values for the 25^{th} percentile of the control values. The lower dotted line in (F) indicates 40 mg/dl. (A) HDL particles; (B) large HDL particles; (C) medium HDL particles; (D) small HDL particles; (E) ApoAl; (F) HDL-C.

FIG. 2. LDL-associated lipid parameters in VTE and controls by gender. Solid thick lines indicate mean values and the dotted lines indicate values for the 75^{th} percentile of the control values. The upper dotted line in (F) indicates 160 mg/dl. (A) LDL particles; (B) IDL particles; (C) large LDL particles; (D) small LDL particles; (E) ApoB; (F) LDL-C; (G) ApoB/ApoAl; (H) LDL-C/HDL-C.

FIG. 3. High density lipoprotein cholesterol (HDL-C) levels and HDL particle concentrations for three groups of male subjects: controls (n=49), VTE patients with normal CETP Arg451 genotype (n=41), and VTE patients carrying two relatively rare CETP linked variants, Pro373 and Gln451 (n=8). Among the first two groups of subjects, those carrying the normal CETP Ala373/Arg451 genotype are indicated by open circles while three subjects (one control and two VTE patients) carrying the rare Pro373 variant but the normal Arg451 are indicated by “X”. The solid lines indicate the mean level and the dotted line indicates the 25^{th} percentile of the control group.

FIG. 4. Apolipoprotein Al in female VTE and controls. Solid lines indicate mean values.

FIG. 5. Kaplan-Meier Estimates of the Probability of Recurrent Venous Thromboembolism among Patients with Apolipoprotein Al Levels equal to or greater than the 1.3 mg/ml (67^{th} percentile of thrombosis patients) and among Patients with Lower Levels. The 67^{th} percentile of Apolipoprotein Al was 1.22 (mg/ml) in men and 1.35 (mg/ml) in women.

FIG. 6. Apolipoprotein Al levels (Panel A), High Density Lipid Cholesterol Levels (Panel B), and Large High Density Lipoprotein Levels (Panel C) among Men and Women with and without Recurrent Venous Thromboembolism. The boxes indicate values from the 25^{th} to the 75^{th} percentile, the solid line indicates the median and the whiskers indicate the 5^{th} and 95^{th} percentiles. A. Apolipoprotein Al (n=772, total cohort); B. High Density Lipoprotein Cholesterol (n=396, nested cohort data); C. Large High Density Lipoprotein particles (n=396, nested cohort data).

FIG. 7. HDL-subpopulations and HDL parameters in male VTE patients and controls. Solid thick lines indicate mean values and the dotted lines indicate values for the 25th percentile of the control values. The lower dotted line in (F) indicates 40 mg/dl. (A) HDL particles; (B) large HDL; (C) medium HDL; (D) small HDL; (E) ApoAl; (F) HDL-C.

FIG. 8. LDL-subpopulations and LDL parameters in male VTE patients and controls. Solid thick lines indicate mean values and the dotted lines indicate values for the 75th percentile of the control values. The upper dotted line in (F) indicates 160 mg/dl. (A) LDL particles; (B) IDL; (C) large LDL; (D) small LDL; (E) ApoB; (F) LDL-C; (G) ApoB/ApoAl ratio; (H) LDL-C/HDL-C ratio.

### DETAILED DESCRIPTION OF THE INVENTION

The present invention is based on the discovery of novel risk factors associated with venous thrombosis. In particular, the present invention is based on the finding that venous thrombosis is associated with decreased levels of protective large HDL particles and, for males, elevated levels of harmful small LDL particles and IDL particles. Genetic studies show that males with venous thrombosis disproportionately carry CETP alleles that convey elevated CETP mass and activity that likely contributes to the dyslipoproteinemia observed in male VTE patients.

In one embodiment, the present invention provides a method of determining an individual at risk for venous thrombosis comprising measuring a level of HDL-C, HDL particles, large HDL particles (HDL2), CETP, apolipoprotein Al and/or apolipoprotein ClII in a test biological specimen obtained from an individual and comparing the level of the lipid or lipoproteins in the test biological specimen to a normal range of lipid or lipoprotein in a normal biological specimen, where a deficiency or lower level of HDL-cholesterol, large HDL particles (HDL2), apolipoprotein Al, and/or apolipoprotein ClII in the test biological specimen is indicative of a risk factor for venous thrombosis for the individual. In a preferred embodiment for apolipoprotein ClII measurement, the determination of apolipoprotein ClII that is not bound to apolipoprotein B is made. In a preferred embodiment for CETP, the determination of CETP mass or activity is made where an elevated level is indicative of increased risk.

Quantitation of the level of lipid or lipoprotein is determined in comparison with reference standards by techniques well known in the art, including, but not limited to, nuclear magnetic resonance (NMR) spectroscopy, plasma or serum lipid assays, ELISA assays, immuno-turbidmetric assays, radioimmunoassay (RIA), capillary electrophoresis, and two dimensional gel electrophoresis with or without immunodetection method. The biological fluid is preferably, blood, plasma or serum, but can also be derived from lung fluid, saliva, cerebrospinal fluid, lymph, urine, semen, saliva, and the like. As well known in the art, lipid or lipoprotein analytes sometimes differ in concentration in males compared with females.

In one embodiment, where normal range indicates values between the 25^{th} and 75^{th} percentile of normal control subjects, normal male ranges of serum HDL-C, plasma HDL particles and large HDL particles (HDL2), apolipoprotein Al and/or apolipoprotein ClII associated with HDL are 43-61.5 mg/dl, 23.9-28.9 μM, 2.55-6.7 μM, 0.9-1.17 mg/ml, and 0.077-0.10 mg/ml, respectively. Considered to constitute below normal values are values of these HDL-related parameters that are below the 25^{th} percentile of normal. In another embodiment, lipid parameters considered below normal level are levels of serum HDL-C below 50 mg/dl, and levels of plasma HDL particles, large HDL particles (HDL2) and apolipoprotein Al below the 35^{th}, percentile of an established normal range or below the 33^{rd} percentile of an established normal range. In another embodiment, apolipoprotein ClII that is not associated with apolipoprotein B below the 15^{th} percentile of an established normal range is considered a low value. In another embodiment, preferentially below-normal levels of lipid parameters considered are levels of serum HDL-C below 45 mg/dl, and levels of plasma HDL particles, large HDL particles (HDL2) and apolipoprotein Al below the 25^{th} percentile of an established normal range. Further, a level of apolipoprotein ClII that is not associated with apolipoprotein B below the 10^{th} percentile of an established normal range is considered a low value. Considered to constitute deficiencies are plasma or serum HDL particles, large HDL particles (HDL2) and apolipoprotein Al below the 35^{th} percentile of the normal mean range or below the 33^{rd} percentile of an established normal range, and/or plasma or serum apolipoprotein ClII that is not associated with apolipoprotein B below the 15^{th} percentile of the normal range. These deficiencies represent plasma concentrations of plasma HDL particles, large HDL particles (HDL2), apolipoprotein Al and/or apolipoprotein ClII not associated with apolipoprotein B in males of about or less than 25.4 μM, 3.4 μM, 0.966 mg/ml, and 0.073 mg/ml, respectively. Further to this embodiment, preferentially deficiencies are below the 25^{th} percentile of the normal mean range for plasma HDL particles, large HDL particles (HDL2) and apolipoprotein Al, and/or below the 10^{th} percentile of the normal mean range for apolipoprotein ClII not associated with apolipoprotein B. For one embodiment, the plasma concentrations for deficiencies for plasma HDL particles, large HDL particles (HDL2), apolipoprotein Al and apolipoprotein ClII not associated with apolipoprotein B in males are of about or less than 23.9 μM, 2.55 μM, 0.90 mg/ml, and 0.070 mg/ml, respectively.

In another embodiment, the present invention provides a method of determining an individual's risk for venous thrombosis comprising measuring a level of LDL-C or LDL particles in a test biological specimen obtained from an individual, and comparing the level of the LDL-C or LDL particles, including, but not limited to, IDL and/or small LDL particles, to a normal range of LDL-C or LDL particles from a biological specimen, where an increase or higher than normal level of LDL-C or LDL particles in the test biological specimen compared to a normal biological specimen is indicative of a risk factor for venous thrombosis for the individual.

In one embodiment, where normal range indicates serum values between the 25^{th} and 75^{th} percentile of normal control subjects, normal ranges of serum LDL-C, plasma LDL particles, IDL particles, small LDL particles and apolipoprotein B in males are 100-140 mg/dl, 750-1175 nM, 948 nM, 440-927 nM and 0.65-0.92 mg/ml, respectively. Considered to constitute above normal values associated with increased risk for thrombosis are values of these LDL-related parameters that are above the 75^{th} percentile of normal. In another embodiment, lipid parameters considered above normal level associated with increased risk for thrombosis are levels of serum LDL-C above 140 mg/dl, and levels of plasma LDL particles, IDL particles, small LDL particles and apolipoprotein B above the 65^{th} percentile of the normal mean range. Further to this embodiment, above normal levels of lipid parameters considered are levels of serum LDL-C above 160 mg/dl, and levels of plasma LDL particles, IDL particles, small LDL particles and apolipoprotein B above the 75^{th} percentile of the normal range.

In another embodiment, increased risk of thrombosis is associated with thrombosis in subjects with elevated lipid or lipoprotein plasma or serum levels which are defined as plasma LDL particles, IDL particles, small LDL particles and/or apolipoprotein B above the 65^{th} percentile of the normal mean range. In one embodiment, excessive or elevated levels represent plasma and/or serum concentrations in males of, about or more than 1130 nM, 35 nM, 810 nM and 0.87 mg/ml, respectively. In a further embodiment, elevated lipid or lipoprotein levels associated with increased risk for thrombosis are defined as above the 75^{th} percentile of the normal mean range for plasma or serum LDL particles, IDL particles, small LDL particles, and/or apolipoprotein B. These elevated levels represent plasma and/or serum concentrations in males of about or more than 1170 nM, 48 nM, 925 nM and 0.92 mg/ml, respectively.

In another embodiment, the present invention provides a method of determining an individual's risk for venous thrombosis comprising measuring the levels of apolipoprotein B and apolipoprotein Al or of LDL-C and HDL-C in a test biological specimen obtained from an individual, determining the ratio of apolipoprotein B to apolipoprotein Al or the ratio of LDL-C to HDL-C, and comparing the test subject's ratio for a biological specimen to the range of normal ratios, where an increase or higher than normal ratio of apolipoprotein B to apolipoprotein Al or of LDL-C to HDL-C in the test biological specimen compared to a normal biological specimen is indicative of a risk factor for venous thrombosis for the individual. In one embodiment, the biological specimen is plasma or serum.

Plasma or serum HDL-C and LDL-C or apolipoprotein Al and apolipoprotein B are determined using methods well known to those skilled in the art, and ratios of values are calculated. The normal range is based on values of ratios that are determined for normal subjects. Ratio values above the normal range are indicative of increased risk of thrombosis. In one embodiment, the normal range values for the ratio of apolipoprotein B to apoliprotein Al and for the ratio of LDL-C to HDL-C are 0.59-0.96 and 1.7-2.9, respectively. In another embodiment, normal range values for the ratio of apolipoprotein B to apoliprotein Al and for the ratio of LDL-C to HDL-C are 0.70-0.85 and 2.0-3.5, respectively. In another embodiment, if the ratio of apolipoprotein B to apoliprotein Al or the ratio of LDL-C to HDL-C is above the 65^{th} percentile of the normal range, it is considered above normal levels and is indicative of increased risk of thrombosis. Values of ratios above the 65^{th} percentile of the normal range for the ratio apolipoprotein B to apoliprotein Al or for the ratio of LDL-C to HDL-C are about 0.83 and 2.6, respectively. In another embodiment, if the ratio of apolipoprotein B to apoliprotein Al or the ratio of LDL-C to HDL-C is above the 75^{th} percentile of the normal range, it is considered above normal and is indicative of increased risk of thrombosis. Values of ratios above the 75^{th} percentile of the normal range for the ratio apolipoprotein B to apoliprotein Al or for the ratio of LDL-C to HDL-C are about 0.96 and 2.9, respectively. In another embodiment, if the ratio of apolipoprotein B to apoliprotein Al or the ratio of LDL-C to HDL-C is above the 85^{th} percentile of the normal range, it is considered above normal and is indicative of increased risk of thrombosis. Values of ratios above the 75^{th} percentile of the normal range for the ratio apolipoprotein B to apoliprotein Al or for the ratio of LDL-C to HDL-C are about 1.05 and 3.25, respectively.

A variety of protocols that are known in the art including ELISA, radioimmunoassy (RIA), electrophoresis, HPLC, FACS, immuno-turbidometric assays, capillary electrophoresis, and two dimensional gel electrophoresis with or without immunodetection methods can be used as alternatives to NMR spectroscopy for measuring lipids or lipoproteins and provide a similar basis for detection and/or diagnosing altered or abnormal levels of lipids or lipoproteins in blood samples. Normal or standard values for lipids or lipoproteins are established by defining a normal or control range for the parameter being determined using a collection of individual blood, plasma or serum samples or other specimens taken from normal mammalian subjects, preferably human, with antibody to the lipids or lipoproteins under conditions suitable for complex formation. Normal or standard values for lipids or lipoproteins are alternatively established by using a pool of blood, serum or plasma samples taken from normal mammalian subjects, preferably humans. The amount of lipids or lipoproteins can be quantified by various methods, but preferably by photometric means. For immunoassay protocols, antibodies to the lipids or lipoproteins under suitable conditions form complexes which can be quantified by various methods including photometric, colorimetric or turbidometric means or a like method. Quantities of the lipids or lipoproteins expressed in subject samples, control samples and test samples from blood samples are compared with the standard control values. Deviation between control or standard values and subject values establishes the parameters for diagnosing a lipid or lipoprotein deficiency or an above normal level, thereby determining an individual at risk of venous thrombosis. This determination is useful for decisions involving the initiation or continutation of antithrombotic therapies and for monitoring therapeutic treatment. As known to those of skill in the art, some lipid or lipoprotein levels differ by gender and may require gender-specific normal or standard reference values.

A higher or lower level than normal of the lipid or apolipoprotein or lipoprotein particle as described herein in a biological specimen, in particular, blood, is a risk factor for venous thrombosis. Venous thrombosis can occur in association with stroke, surgery, trauma, cancer, leg paresis, prolonged travel, inflammatory bowel disease, Bechet's disease, bone fracture, chemotherapy use, diabetes, or in subjects carrying a genetic risk factor for thrombophilia such as the presence of factor V Leiden or of protein C deficiency or protein S deficiency or antithrombin deficiency, and the like.

Another embodiment of the present invention provides a method of determining an individual's risk for venous thrombosis comprising determining the CETP genotype, including, for example, the B1 or B2 allele of the CETP TaqI polymorphism. The absence of the B2 allele or the presence of the B1 allele in the test biological specimen is indicative of a risk factor for venous thrombosis for the individual. Other polymorphisms in the CETP gene that are in linkage disequilibrium with the B1 or B2 alleles can also be used. Determination of the B1 (wild-type or predominant) or B2 (variant or less frequent) allele of the CETP TaqI B polymorphism is dependent on the nucleotide present at the 277^{th} nucleotide of intron 1 of the CETP gene (Drayna D et al, Nucl Acids Res 1987; 15: 4698). A guanidine “G” at this position defines the B1 allele and an adenine “A” defines the B2 allele.

In a preferred embodiment, the present invention further provides a method of determining an individual's risk for venous thrombosis comprising determining the presence of CETP-linked gene variants, Ala373 to Pro and Arg451 to Gln. The presence of either one or both of these variants in the test biological specimen is indicative of a risk factor for venous thrombosis for the individual.

A variety of protocols to detect the CETP polymorphisms known to those in the art can be used. These protocols involve the use of genetic material (DNA or RNA) from the human subject derived from a specimen from the test subject, including but not limited to blood, buccal cells, semen, saliva, tissue biopsies, and the like. Polymorphism detection is by techniques well known in the art including, but not limited to, polymerase chain reaction (PCR)—restriction fragment length polymorphisms (including site directed mutagenesis), allele specific PCR or amplification refractory mutation system (ARMS), direct sequencing, real-time PCR, single-stranded conformational polymorphisms (SSCP) or heteroduplex analysis, denaturing gradient gel electrophoresis (DGGE), peptide nucleic acid (PNA) clamping, oligonucleotide ligation, hybridization or extension assays, TaqMan or molecular beacons, high-performance liquid chromatography, and the like.

The ability to assess an individual's risk for venous thrombosis based on the dyslipoproteinemia patterns described herein would enable physicians to find and monitor specific treatments to decrease the risk and/or occurrence of venous thrombosis in individuals at increased risk.

Accordingly, another embodiment of the present invention provides a method of reducing an individual's risk for venous thrombosis comprising administration of a lipid lowering drug or a lipid altering drug in an amount sufficient to reduce the individual's risk for venous thrombosis. Drugs that increase HDL-C and/or large HDL particles (HDL2) may be used to reduce the risk of venous thrombosis. Such drugs that favorably increase HDL include CETP inhibitors. Drugs that reduce the levels of LDL-C and/or of small LDL particles may be used to reduce the risk of venous thrombosis in a subject. Such lipid lowering drugs include, but are not limited to statins and CETP inhibitors. Lipid-altering drugs that may also be used to reduce the risk of venous thrombosis include nicotinic acid, fibrates, bile acid sequestrants or newly discovered agents which can either increase HDL-C or large HDL particles (HDL2) and/or decrease LDL-C and/or decrease small LDL particles.

All the essential materials and reagents required for determining lipid or lipoprotein levels in a sample, or for inhibiting risk of venous thrombosis, or for screening for risk factors may be assembled together in a kit. When the components of the kit are provided in one or more liquid solutions, the liquid solution preferably is an aqueous solution, with a sterile aqueous solution being particularly preferred.

For the detection of lipids or lipoproteins the kit may contain materials for chromatographic separation, such as columns, beads, resins, gel matrices, filters, TLC plate, buffers and appropriate solvents. Alternatively, if the detection is via immunologic means, the kit may contain antibodies directed to the lipids or apolipoproteins, secondary antibodies that bind primary antibodies, labels or signal generating compounds (either conjugated or unconjugated) and various reagents for the generation and detection of signals.

The components of these kits may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet for explaining the assays for determining lipid or lipoprotein levels in samples.

The kits of the present invention also will typically include a means for containing the vials in close confinement for commercial sale such as e.g., injection or blow-molded plastic containers into which the desired vials are retained. Instrumentation may also be included, for example, devices that permit the reading or monitoring of reactions in vitro.

The contents of all published articles, books, reference manuals and abstracts cited herein, are hereby incorporated by reference in their entirety to more fully describe the state of the art to which the invention pertains.

The following examples demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The concentration of each lipoprotein subclass and the levels (serum and antigenic) of apolipoprotein (apo) Al and B were determined using a proton nuclear magnetic resonance spectroscopy (NMR). Single nucleotide polymorphisms (SNPs) were analyzed in three key genes that influence lipoprotein metabolism and subpopulation levels, hepatic lipase, endothelial lipase and cholesterol ester transfer protein (CETP) (16-20).

### Methods

Study Group: The Scripps Venous Thrombosis Registry is an ongoing case-control study of risk factors for VTE. Patients with objectively documented VTE were recruited from the Scripps Anticoagulation Service and the community. Identification of novel genetic risk factors for VTE is a major goal for the Registry, and genetic factors are more likely to contribute to VTE in younger subjects under 55 years old. Inclusion criteria for this study included age at thrombosis <55 years,> 3 months since diagnosis of acute thrombosis, a life expectancy of at least three years and no lipid lowering medications or metastatic cancer. Age and sex matched healthy controls were recruited through the Scripps General Clinical Research Center's (GCRC) blood-drawing program. Clinical data collection included detailed medical history and the presence of risk factors for venous thrombosis. The protocol was approved by the Institutional Review Board of Scripps Clinic and subjects provided written informed consent.

Clinical characteristics, the frequency of identified risk factors and serum lipid data are shown in Table 1. Fifty-seven of 115 VTE patients (49.6%) presented with idiopathic VTE, defined as events that did not occur within 90 days after surgery, trauma, major immobilization and that were not associated with factor V Leiden or prothrombin 20210A, pregnancy or estrogen use. VTE was more often idiopathic in males (82%) than females (24.6%) (p<0.001). In the 65 female VTE patients, the VTE event was associated with pregnancy in 7 (11%), oral contraceptive use in 25 (39%) and estrogen replacement therapy in 7 (11%). Oral contraceptive use in controls (30%) was similar; estrogen replacement therapy in controls (22%) was higher than in VTE patients. Estrogen use in females with VTE was at time of VTE episode. Eighty-four percent of patients were taking warfarin when blood was donated.

TABLE 1
Study population

| | Total | | | Male | | | Female | | |
|---|---|---|---|---|---|---|---|---|---|
| | Control | VTE | | Control | VTE | | Control | VTE | |
| Variables | N = 115 | N = 115 | p | N = 49 | N = 50 | p | N = 66 | N = 65 | p |
| Age (yr ± SD) | 45.5 (9.4) | 45.4 (10.0) | 0.95 | 46.3 (5.2) | 47 (8.9) | 0.66 | 44.8 (10.2) | 44.1 (10.7) | 0.68 |
| Gender, no. (%) | | | | | | | | | |
| Male Sex | 49 (42.6) | 50 (43.5) | 1.0 | | | | | | |
| Female Sex | 66 (57.4) | 65 (56.5) | | | | | | | |
| Ethnic Group (%) | | | | | | | | | |
| Non-Hispanic White | 93 | 93 | 1.0 | 91.8 | 92 | 0.52 | 93.9 | 93.8 | 1.0 |
| Body-mass Index | 27.1 (5.5) | 29.3 (6.5) | 0.006 | 27.2 (4.3) | 30.6 (5.9) | 0.001 | 27.1 (6.3) | 28.4 (6.8) | 0.29 |
| (kg m⁻²) | | | | | | | | | |
| Risk factors, | | | | | | | | | |
| no. (%) | | | | | | | | | |
| Previous thrombosis | | 37 (32.2) | | | 19 (38) | | | 18 (27.6) | |
| Family History | 12 (10.4) | 44 (38.3) | <0.0001 | 4 (8.1) | 18 (36) | 0.001 | 8 (12.1) | 26 (40.0) | 0.0003 |
| Estrogen Use in | | | | | | | | | |
| Females | | | | | | | | | |
| Oral contracep- | | | | | | | 20 (30.3) | 25 (38.5) | 0.36 |
| tives | | | | | | | | | |
| Estrogen replace- | | | | | | | 14 (21.5) | 7 (10.7) | 0.15 |
| ment | | | | | | | | | |
| Pregnancy | | | | | | | | 7 (10.7) | |
| Factor V Leiden | 6 (5.3) | 28 (24.6) | <0.0001 | 2 (4.1) | 13 (26.0) | 0.002 | 4 (6.2) | 14 (21.9) | 0.01 |
| mutation | | | | | | | | | |
| Prothrombin gene | 4 (3.5) | 9 (7.9) | 0.15 | 2 (4.1) | 7 (14.0) | 0.09 | 2 (3.1) | 2 (3.1) | 0.99 |
| mutation | | | | | | | | | |
| Idiopathic | | 57 (49.6) | | | 41 (82.0) | | | 15 (24.6) | |
| Clinical Lipids | | | | | | | | | |
| (Mean ± SD) | | | | | | | | | |
| total cholesterol | 197 (40) | 208 (41) | 0.05 | 205 (41) | 216 (43) | 0.15 | 191 (38) | 200 (39) | 0.14 |
| (mg/dl) | | | | | | | | | |
| Triglyceride | 124 (72) | 132 (84) | 0.47 | 143 (84) | 159 (96) | 0.35 | 110 (59) | 110 (67) | 0.94 |
| (mg/dl) | | | | | | | | | |
| HDL cholesterol | 57.3 (16.4) | 54.3 (16.4) | 0.19 | 53.6 (15.0) | 46.3 (17.5) | 0.03 | 60.1 (17.0) | 60.5 (16.7) | 0.89 |
| (mg/dl) | | | | | | | | | |
| LDL cholesterol | 114 (33) | 126 (37.3) | 0.01 | 122 (33) | 135 (40) | 0.05 | 109 (31) | 119 (33) | 0.09 |
| (mg/dl) | | | | | | | | | |
| LDL-C/HDL-C ratio | 2.17 (0.86) | 2.59 (1.18) | 0.002 | 2.45 (0.91) | 3.19 (1.22) | 0.001 | 1.96 (0.75) | 2.12 (0.91) | 0.26 |

Blood collection, lipids and apolipoproteins: Blood was collected in the GCRC at least three months after VTE diagnosis and after 12 hours fasting. Serum and EDTA-plasma were prepared and plasma was stored at −70° C. Plasma levels of ApoAl and ApoB were measured using immunoturbidometric assay kits (DiaSorin, Stillwater, Minn.). Serum lipid profile data were obtained from the routine clinical lab using standard techniques.

NMR lipoprotein subclass analysis: Lipoprotein particle concentrations of 10 lipoprotein subclasses were determined in EDTA plasma by proton NMR spectroscopy (21) at LipoScience (Raleigh, N.C.). The subclass categories based on particle diameter range comprised: 3 VLDL subpopulations [chylomicron/large VLDL, intermediate VLDL, and small VLDL]; 3 LDL subpopulations [IDL, large LDL, and small LDL which was also reported as medium small LDL and very small LDL]; and 3 HDL subpopulations [large HDL, medium HDL and small HDL]. Values for mean VLDL, LDL, and HDL particle size were also calculated. As previously emphasized (21, 22), the NMR-derived lipoprotein particle levels are based on the NMR signals that are characteristic of typical lipoprotein particles and are not actual lipid measurements. NMR data are directly proportional to the number of particles, independent of lipid or apolipoprotein per particle which may vary from person to person.

DNA analysis: Genomic DNA was extracted from EDTA-blood using Puregene® DNA Purification Kits (Gentra Systems, Minneapolis, Minn.). Factor V Leiden and prothrombin 20210A SNPs and hepatic lipase (LIPC-514C/T), endothelial lipase (LIPG T111I) and CETP (TaqI B and 1405V) SNPs were assayed as described (17, 20, 23-25).

Statistical analysis: Categorical data between groups were compared using contingency-table analysis (chi-squared test), and continuous data were compared using either unpaired student t-test or Mann-Whitney as appropriate (Prism™ 3.0 software, Graph Pad Software, San Diego, Calif.). All p-values were two-tailed. The multifactorial role of prothrombotic risk factors was assessed using multivariate logistic regression analysis, and adjusted p-values or adjusted odds ratio' were calculated using Minitab14 software (Minitab, State College, Pa.).

### Results

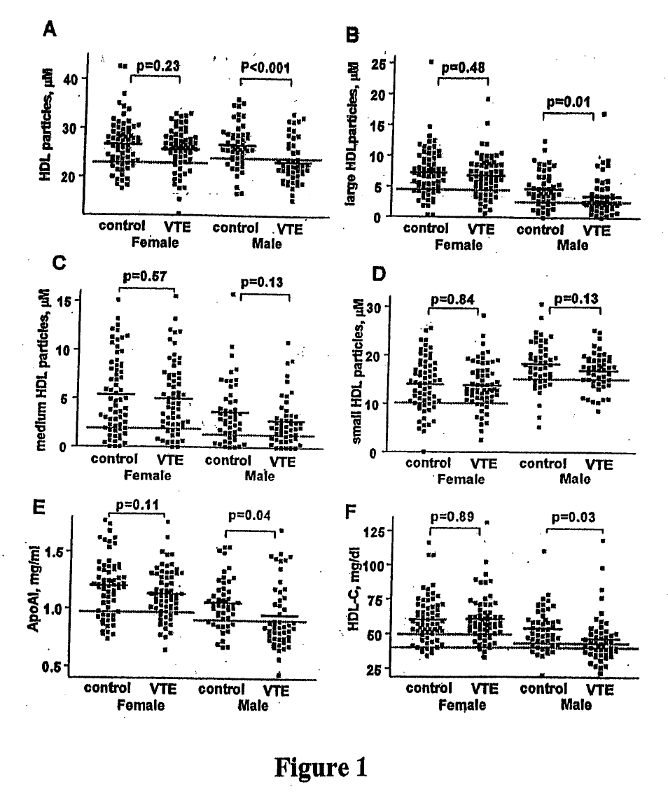

Male VTE cases had a significantly lower mean HDL particle concentration than male controls (p<0.001) (FIG. 1A). Among HDL lipoprotein subclasses, large HDL particle concentrations were lower in male VTE patients than male controls (p=0.01), whereas medium and small HDL particles were not significantly different (p=0.13 and 0.13, respectively) (FIG. 1B-D). The HDL particle size was also smaller in male VTE cases than male controls (p=0.02) (data not shown).

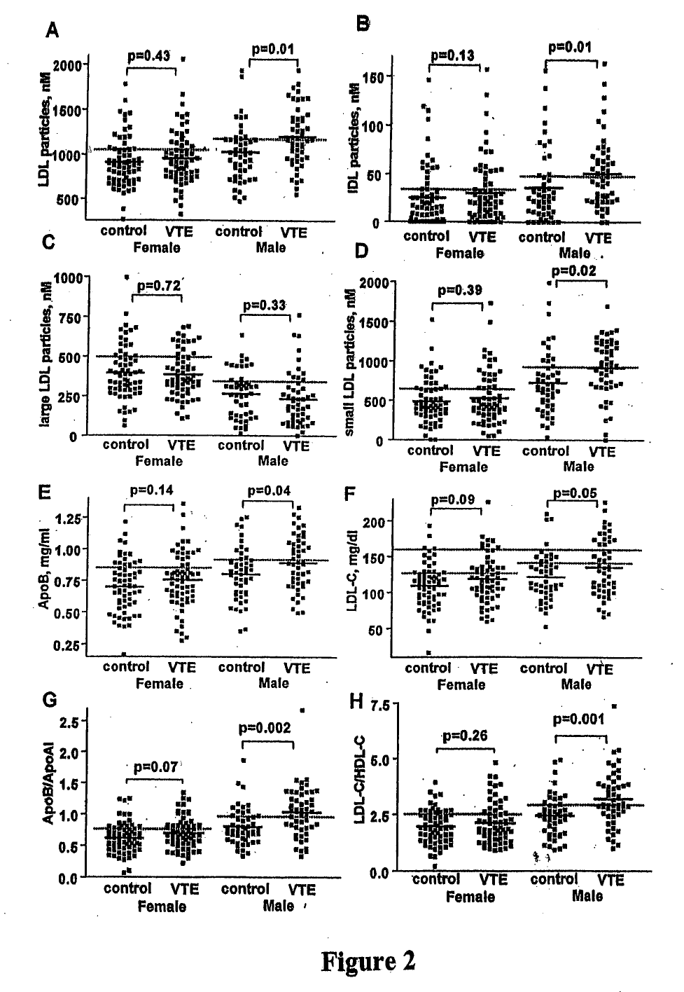

LDL lipoprotein particle concentrations in male VTE patients were significantly higher than in male controls (FIG. 2A, p=0.01). Among LDL lipoprotein subclasses, IDL and small LDL particle concentrations were higher in males with VTE (p=0.01 and 0.02, respectively), whereas large LDL particle levels showed no difference (p=0.33) (FIGS. 2B-D). The two subgroups of small LDL particles, namely medium small particles and very small LDL particles, were elevated in male VTE cases (p=0.01 and 0.02, respectively, not shown). The LDL particle size was smaller in male VTE cases compared to male controls (p=0.04) (not shown). After adjustments for three well-recognized risk factors for venous thrombosis, factor V Leiden, prothrombin 20210A, and body mass index (BMI), strong statistical significance remained for males for differences in concentrations of HDL particles, large HDL particles, LDL particles, and small LDL particles with adjusted p values of 0.004, 0.04, 0.01 and 0.02, respectively. The adjusted p value for the difference in IDL particles became less significant and was 0.07.

In contrast to male subjects' data, no significant differences in HDL or LDL particle concentrations were seen when female VTE patients were compared to female controls (FIGS. 1 and 2). For both males and females, no statistically significant differences were observed for VLDL particle total concentration or for VLDL subclasses (large, medium and small VLDL particles) or for VLDL particle size (data not shown).

ApoAl levels were lower (p=0.04) (FIG. 1E) and apoB levels were higher (p=0.04) in male VTE patients compared to male controls (FIG. 2E). The level of apolipoprotein ClII that was not associated with apolipoprotein B (apoClII-Lp-nonB) was also lower in VTE (p=0.03) (data not shown). The mean value for the apoB to apoAl ratio for male VTE patients was significantly higher than that for male controls (p=0.001) (FIG. 2G). This difference in the apoB/apoAl ratio mean value was statistically stronger than the differences for the mean values of either ApoAl alone or ApoB alone when VTE patients were compared to controls (FIG. 2G vs FIGS. 1E and 2E). After adjustment for factor V Leiden, prothrombin 20210A, and BMI, the adjusted p value was 0.008. No differences in apoAl or apoB mean values were seen in comparisons of female VTE patients to female controls.

Total serum cholesterol and triglyceride levels showed no differences between the mean values for VTE and control groups for either gender (Table 1). For male VTE patients compared to male controls, the mean value of HDL-C was lower (p=0.03) while the mean value of LDL-C was higher (p=0.05). Remarkably however, the LDL-C to HDL-C mean ratio was very significantly higher for VTE males compared to male controls (p=0.002) (FIG. 2H). After adjustment for factor V Leiden, prothrombin 20210A, and BMI, the adjusted p value for the difference in ratio mean values was 0.002.

Quartile-based odds ratios (OR) for reduced HDL parameters (<25%) and elevated LDL parameters (>75%) are shown for VTE in males in Table 2. Low levels of total HDL particles and of large HDL particles are associated with increased VTE risk, OR=7.2 (95% Cl: 3.0-18) and 3.3 (95% Cl: 1.4-7.9), respectively. Smaller HDL average particle size had a significant odds ratio of 3.1 (95% Cl: 1.3-7.3). High levels (>75% ile) of total LDL particles and of two subfractions, IDL and small LDL particles, increase the risk of VTE [OR=2.9 (95% Cl: 1.2-6.7), 2.6 (95% Cl: 1.1-6.2) and 3.6 (95% Cl: 1.5-8.5), respectively]. For VTE in males, two subpopulations of the small LDL particle subclass, i.e., medium small and very small LDL particles, each gave an OR of 3.3 (95% Cl: 1.4-7.9 and 1.5-8.5, respectively, data not shown), a value similar to the OR=3.6 for small LDL particles (Table 2). After adjustment for factor V Leiden, prothrombin 20210A, and BMI, all statistically significant quartile-based OR values retained strong statistically significance (data not shown).

TABLE 2
Odds ratio of VTE for LDL or HDL associated lipid parameters
in males based on quartile analyses

| | | | |
|---|---|---|---|
| Variables | OR | (95% CI) | p-value |

HDL associated variables

| | | | |
|---|---|---|---|
| HDL particles | 7.2 | (3.0-18) | <0.0001 |
| large HDL | 3.3 | (1.4-7.9) | 0.006 |
| medium HDL | 1.3 | (0.54-3.2) | 0.54 |
| small HDL | 1.6 | (0.66-3.8) | 0.30 |
| HDL size | 3.1 | (1.3-7.3) | 0.01 |
| Apo AI | 5.5 | (2.3-13) | 0.0005 |
| HDL cholesterol | 3.3 | (1.4-7.9) | 0.006 |

LDL associated variables

| | | | |
|---|---|---|---|
| LDL particles | 2.9 | (1.2-6.7) | 0.02 |
| large LDL | 2.6 | (1.1-6.2) | 0.03 |
| medium LDL | 0.87 | (0.34-2.2) | 0.77 |
| small LDL | 3.6 | (1.5-8.5) | 0.003 |
| LDL size | 1.9 | (0.63-5.7) | 0.26 |
| Apo B | 2.4 | (1.0-5.7) | 0.04 |
| LDL cholesterol | 2.2 | (0.94-53) | 0.07 |

Combination ratios

| | | | |
|---|---|---|---|
| LDL-C/HDL-C | 3.3 | (1.4-7.9) | 0.006 |
| Apo/B/ApoAI | 3.9 | (1.7-9.3) | 0.002 |

The odds ratios (OR) for VTE in male subjects based on the levels of HDL-associated variables below the 25^{th} percentile of controls or based on the levels of LDL-associated parameters above the 75^{th} percentile of controls were calculated. 95% CI denotes the 95 percent confidence interval.

In males, low levels of apoAl and high levels of apoB gave significant OR values for increased risk of VTE, OR=5.5 (95% Cl: 2.3-13) and 2.4 (95% Cl: 1.0-5.7), respectively, as did the ratio of ApoB/ApoAl [OR=3.9 (95% Cl: 1.7-9.3)]. Low levels of apoClII-Lp-non-B (below the 10^{th} percentile of controls) were associated with increased VTE risk [OR=3.1 (95% Cl: 1.0-9.5), p=0.04] (data not shown). Based on males' clinical lab serum cholesterol data, the OR for low HDL-C was statistically significant [OR=3.3 (95% Cl: 1.4-7.9)] while for elevated LDL-C, although it was elevated, it did not achieve statistical significance [OR=2.2 (95% Cl: 0.94-5.3)]. For male VTE, a high ratio of LDL-C/HDL-C was statistically significant for increased VTE risk [OR=3.3 (95% Cl: 1.4-7.9)]. For male LDL-C levels above 160 mg/dl, the OR for VTE was 4.8 (95% Cl: 1.5-16) while for HDL-C below 40 mg/dl, the OR for VTE was 3.1 (95% Cl: 1.2-8.1) (data not shown).

To identify genetic influences contributing to dyslipoproteinemia, well known SNPs in three key genes that influence lipoprotein subpopulations and HDL-C, hepatic lipase (LIPC-514C/T), endothelial lipase (LIPG T111I) and CETP (TaqI B and 1405V), were determined (Table 3). Using chi-squared analysis, the allele frequencies of the SNPs did not differ between all VTE patients and the control group. However, the CETP B2 allele was significantly less common in male VTE cases than controls (p=0.04), and the CETP B2 allelic difference gave an OR of 1.9 (95% Cl: 1.1-3.4).

TABLE 3
Allele frequencies of the less common allele for polymorphisms
in hepatic lipase (LIPC), endothelial lipase (LIPG) and cholesteryl
ester transfer protein (CETP) in VTE cases and controls

| | Total | | | Men | | | Women | | |
|---|---|---|---|---|---|---|---|---|---|
| | | Con- | | | Con- | | | Con- | |
| | VTE | trols | P | VTE | trols | P | VTE | trols | P |
| LIPC- | 0.24 | 0.25 | 0.86 | 0.24 | 0.21 | 0.67 | 0.23 | 0.27 | 0.52 |
| 514C/T | | | | | | | | | |
| LIPG | 0.31 | 0.32 | 0.84 | 0.28 | 0.33 | 0.48 | 0.34 | 0.32 | 0.73 |
| T111I | | | | | | | | | |
| CETP | 0.39 | 0.47 | 0.06 | 0.32 | 0.47 | 0.04 | 0.44 | 0.48 | 0.53 |
| TaqI | | | | | | | | | |
| B1/B2 | | | | | | | | | |
| CETP | 0.29 | 0.34 | 0.27 | 0.22 | 0.31 | 0.17 | 0.34 | 0.36 | 0.77 |
| I405V | | | | | | | | | |

### Discussion

Although arterial thrombosis and cardiovascular disease are clearly associated with dyslipoproteinemia (26-31), dyslipoproteinemia in VTE is not known to those of skill in the art. In this example, male VTE patients have markedly lower total HDL particle concentrations and higher LDL particle levels than male controls. Lipoprotein subclass analyses show that these differences reflect lower levels of large HDL particles and higher levels of IDL and of small LDL particles. Confirming the NMR-based demonstration of dyslipoproteinemia in male VTE patients, antigenic assay data for the major proteins of HDL and LDL showed lower apoAl and elevated apoB levels. Differences in ApoB/ApoAl ratios between male VTE patients and controls were statistically stronger than differences in either apolipoprotein alone. In this example, all statistically significant differences for lower HDL parameters and elevated LDL parameters between VTE cases and controls were limited to only males compared to females. This finding is related to the much lower percentage of female patients who had idiopathic VTE (25%) compared to males (82%). The power of our study of female VTE patients with idiopathic VTE is not sufficiently strong enough to rule out the possibility that risk of idiopathic VTE in females is associated with one or more elements of the pattern of dyslipoproteinemia that is clearly manifested in our male VTE patients. As shown in Examples 3 and 4, low levels of HDL are associated with increased risk for VTE in females who experience unprovoked VTE.

In this example, based on quartile analyses, the OR for VTE associated with low levels of HDL particles was quite high, being 7.2 (95% Cl: 3.0-18), and it was associated specifically with low levels of large HDL particles and not with differences in medium and small HDL particles. Statistically significant OR values were found for male VTE associated with elevated LDL particle concentrations due to elevations of small LDL particles and IDL particles but not large LDL particles.

Based on serum cholesterol data, HDL-C was significantly lower in male VTE cases than controls, and the ratio of LDL-C/HDL-C was significantly higher in VTE patients than controls. Although LDL-C data were not particularly striking, the OR for VTE in subjects with LDL-C level above 160 mg/dl was 4.8 (95% Cl: 1.5-16) suggesting that elevated LDL-C is associated with VTE. When the quartile-based OR for VTE associated with elevated LDL-C was calculated, the OR did not achieve statistical significance.

To identify genetic factors contributing to the observed dyslipoproteinemia, genetic variation was assessed in three genes regulating HDL metabolism (17, 18, 20, 32, 33). The B2 allele of the CETP TaqI polymorphism was less frequent in male (p=0.04) VTE cases compared to controls. CETP plays a pivotal role in cholesteryl ester transfer from HDL to ApoB-containing lipoproteins as CETP deficiency or CETP inhibitors increase HDL levels (33, 34). The TaqI B2 allele is linked to decreased CETP activity that causes larger HDL particle size and LDL particle size (16) such that lower B2 frequency in VTE would predict lower HDL and higher LDL levels. Of note, the CETP TaqI locus is in strong linkage disequilibrium with other polymorphisms in the CETP gene that may directly affect CETP activity and concentration (35). Thus, the TaqI B polymorphism provides only one of several SNP markers of the CETP genotype that is linked to increased risk for venous thrombosis in males. Each CETP SNP associated with VTE is linked to higher levels of CETP mass and activity, indicating that the genetic studies indicate that higher levels of CETP mass and activity are linked to VTE risk.

Recurrent VTE occurs more often in men than women, and this was not explained by any obvious factors (36). This increased risk for VTE recurrence and the intrinsically higher VTE risk for males versus females (37) suggest that there are risk factors specific to men and/or protective factors specific to women. In general, premenopausal women have more favorable lipid/lipoprotein profiles than men. This discovery helps to explain why males are at greater risk for VTE. As reviewed elsewhere (13), elevated LDL or oxidized LDL can promote thrombin formation whereas HDL can enhance the protein C pathway and reduce thrombin generation. Thus, an increased ratio of LDL to HDL, reflected in apoB/apoAl or LDL-C/HDL-C values, could be prothrombotic, and there is substantial biologic plausibility for mechanisms by which dyslipoproteinemia can be prothrombotic for VTE.

Based on the findings that dyslipoproteinemia is associated with VTE in this example and in the other examples, there are obvious therapeutic and diagnostic implications. Implications for novel therapies include, but are not limited to, the following. Because risk for VTE is higher in subjects with the dyslipoproteinemia pattern described here, such subjects can benefit from therapies that favorably alter the dyslipoproteinemia. For a patient who experienced unprovoked VTE, also known as idiopathic VTE, thromboprophylaxis with lipid altering drugs will prevent recurrent VTE. In subjects without a previous VTE who present with this dyslipoproteinemia pattern as a VTE risk factor, statin therapy or CETP therapy can be effective for decreasing VTE risk for a first event during situations of increased risk. Clinical trials show that CETP inhibitors cause increases in levels of large HDL particles and decreased levels of LDL particles, i.e., a favorable reversal of the dyslipoproteinemia pattern seen in VTE patients (34), suggesting that CETP inhibitor therapy is useful for reducing risk of recurrent VTE in patients with dyslipoproteinemia. CETP inhibitor will be useful to prevent VTE in subjects with HDL deficiency who would benefit from prophylaxis whether or not they have had a prior VTE.

Diagnostic applications include, but are not limited to, the following: For example, LDLC/HDL-C ratio can be clinically useful to assess VTE risk. Although less convenient than serum lipid assays, ELISA assays or other immunoassays known to those of skill in the art to determine apoB/apoAl ratios can also be useful. In addition, further clinical studies using NMR spectroscopy to quantitate lipoprotein subclass levels in VTE patients are well warranted as are studies of SNPs in genes that regulate HDL and LDL metabolism. SNPs of interest include the CETP TaqI B1/B2 polymorphism or other genetically linked polymorphisms known to those of skill in the art.

The statistical significance of our data is compelling because a number of independently measured parameters, namely NMR data, antigenic data and clinical serum cholesterol data, were completely coincident in defining a particular pattern of dyslipoproteinemia characterized by lower levels of large HDL particles and elevated levels of small LDL and IDL particles in this example.

In summary, in Example 1, VTE in males was associated with decreased levels of protective large HDL particles and elevated levels of harmful small LDL particles and IDL particles. Genetic studies show that males with VTE disproportionately carry CETP genotypes that are associated with elevated CETP activity and that contributes to the dyslipoproteinemia observed in male VTE patients.

### EXAMPLE 2

The prevalence of two relatively rare CETP variants, Ala373Pro and Arg451 Gln, was determined in a cohort of VTE male patients and matched controls. The data showed that rare and usually linked CETP variants, Pro373 and Gln451, were associated with venous thrombosis and low plasma levels of HDL. The CETP variants, Ala373Pro and Arg451Gln, are known to be associated with elevated levels of CETP mass and activity, indicating that elevated CETP mass and activity are linked to VTE.

### Methods

Study group: Male patients with objectively documented VTE (n=49) were recruited into the Scripps Venous Thrombosis Registry (42). Among various considerations, inclusion criteria included age at thrombosis of less than 55 years, greater than 3 months since diagnosis of acute thrombosis, no use of lipid lowering medications, and no cancer (42). Healthy male controls (n=49) were matched by age (±2 years) and ethnic group (92% non-Hispanic white). VTE was classified as idiopathic for 82% (40/49) of cases. The protocol was approved by the Institutional Review Board of Scripps Clinic and subjects provided written informed consent.

Blood collection, lipids, and lipoprotein subclass analysis: Serum and EDTA plasma were prepared from blood collected from patients at least three months after VTE diagnosis and after 12 hours fasting and were stored at −70° C. Serum lipid profile data were obtained from the routine clinical lab using standard techniques.

Lipoprotein particle concentrations of 10 lipoprotein subclasses in EDTA plasma were determined by proton NMR spectroscopy (58,59) (LipoScience (Raleigh, N.C.)), as described (42). The subclass categories based on particle diameter range comprised: 3 VLDL subpopulations [chylomicron/large VLDL, intermediate VLDL, and small VLDL]; 3 LDL subpopulations [IDL, large LDL, and small LDL]; and 3 HDL subpopulations [large HDL, medium HDL and small HDL]. Values for mean VLDL, LDL, and HDL particle size were also calculated. It has been emphasized that the NMR-derived lipoprotein particle levels are based on the NMR signals that are characteristic of typical lipoprotein particles and are not actual lipid measurements (58,59). NMR data directly yield concentrations of particles, independent of lipid or apolipoprotein per particle which may vary from person to person.

DNA analysis: Genomic DNA was extracted from EDTA-blood using Puregene® DNA Purification Kits (Gentra Systems, Minneapolis, Minn.). Four SNPs in the CETP gene (TaqI B1/B2, Ala373Pro, lle405Val, and Arg451Gln) were assayed as described (42,52). To evaluate the association of VTE with genetic polymorphisms, chi-squared analysis was used.

### Results and Discussion

CETP plays a pivotal role in lipoprotein metabolism and potently remodels HDL by transferring various lipids between different lipoproteins, and CETP deficiency or CETP inhibitors increase HDL levels (53-55, 60-62). Four CETP gene variations, namely the relatively common TaqI B1 and lle405 variants and the relatively rare Pro373 and Gln451 variants, have been associated with higher plasma CETP levels that cause lower HDL cholesterol levels and smaller HDL particles (52-57). To gain insight into HDL deficiency in male venous thrombosis patients (42), the Ala373Pro and Arg451Gln variations in the CETP gene were analyzed for 49 male VTE cases and 49 matched controls. Heterozygosity for Pro373 was identified in 20% of VTE cases (10 of 49) and 2% of controls (1 of 49) (p=0.0040) while heterozygosity for Gln451 was identified in 16% of VTE cases (8 of 49) and 0% of controls (p=0.0032). No homozygote for either of these two CETP variants was found among the 98 subjects under study. All subjects carrying Gln451 presented with Pro373. For comparison, Factor V Leiden and prothrombin20210A were found in 26% (13 of 49) and 14% (7 of 49) of VTE cases, respectively, while each of these two genetic variations was found separately in 4% of controls (2 of 49 subjects for each variant), as reported (42).

Allele frequency values for the four studied CETP variations for our Scripps controls (Table 4) esembled published population values (52-57). For example, for Caucasian populations, the published allele frequency values for Pro373 and Gln451 are approximately 0.02 and 0.01, respectively, and those for TaqI B1 and lle405 are 0.57 and 0.62, respectively. In contrast, all allele frequency values for our Scripps VTE patients exceeded values for our Scripps controls for Pro373, Gln451, TaqI B1 and lle405, and the calculated differences in allelic frequencies are shown in Table 4.

TABLE 4
Allelic frequency for four SNPs in the CETP gene for
49 male VTE patients and 49 matched controls.

| | | | Difference |
|---|---|---|---|
| CETP Allele | VTE | Controls | [VTE − Controls] |
| 373Pro | 0.10 | 0.01 | 0.09 |
| 451Gln | 0.08 | 0 | 0.08 |
| Taql B1 | 0.67 | 0.53 | 0.14 |
| 405Ile | 0.80 | 0.69 | 0.11 |

Based on analysis of 7 different CETP SNPs, previous studies inferred the existence of at least fourteen different CETP haplotypes, each with an estimated prevalence of >0.5% (56). Only one of those 14 haplotypes carries Gln451, while 2 of 14 haplotypes carry Pro373. The more prevalent of these two haplotypes carries TaqI B1, Pro373, lle405, and Gln451, the four CETP SNPs determined in our study were found to be in excess in VTE patients compared to controls (Table 4). The differences in allelic frequency between VTE patients and controls were 0.09 for Pro373 and 0.08 for Gln451 (Table 4). The differences in allelic frequencies between VTE patients and controls for the more common TaqI B1 and lle405 variants in our study (42) were 0.14 and 0.11, respectively. Thus, each difference in allelic frequency is quite similar, ranging from 0.08 to 0.14, and we speculate that the 16% of our VTE patients who present with both Pro373 and Gln451 carry the haplotype (56) that includes the common CETP variants, TaqI B1 and lle405, and the relatively rare variants, Pro373 and Gln451.

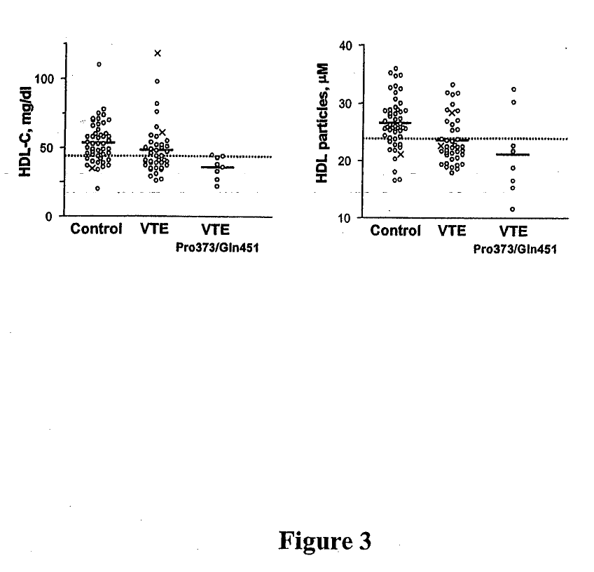

The relationship between plasma lipoprotein concentrations and the presence of Pro373 and Gln451 in 8 VTE patients was analyzed. HDL parameters for those 8 VTE patients were compared to those for both controls and other VTE patients who carried the normal Arg451 CETP genotype. Plasma levels of HDL cholesterol and HDL particles were lower in 8 VTE patients carrying Pro373 and Gln451 than in those with the normal Arg451 CETP genotype (FIG. 3). The VTE subjects carrying both Pro373 and Gln451 genotype did not differ significantly from other subjects in their concentrations of lipoproteins that were determined using NMR spectroscopy, namely, in the concentrations of VLDL particles and LDL particles (data not shown). Thus, we suggest that the presence of Pro373 and Gln451 in CETP in VTE patients significantly helps to explain the finding of HDL deficiency associated with venous thrombosis (42).

It is interesting to note the following. All 8 VTE patients with both Pro373 and Gln451 have HDL cholesterol levels that are ≦the 25th percentile of controls while 6 of 8 of these 8 VTE patients have HDL particle concentrations ≦ the 25th percentile of controls (FIG. 3). For comparison, one notes that 54% of VTE patients with normal CETP genotype have HDL cholesterol levels that are below the 25th percentile of controls while 68% of VTE patients have HDL particle concentrations ≦ the 25th percentile of controls (FIG. 3). These data show that a substantial number of VTE patients with wild-type Ala373. and Arg451 CETP genotypes have low HDL cholesterol and HDL particle concentration (FIG. 3). Consequently, it appears that although the presence of Pro373 and Gln451 significantly helps to explain the finding of HDL deficiency in some VTE patients, there are other factors, genetic and/or environmental, that contribute significantly to HDL deficiency in venous thrombosis.

HDL contributes to reverse cholesterol transport process as well as to potent anti-inflammatory, antioxidant, and anti-apoptotic activities (44-46, 54, 55, 62-66). HDL also enhances the anticoagulant activity of activated protein C, at least in vitro (50). Thus, there is substantial biologic plausibility for pathogenic mechanisms by which HDL deficiency caused by the CETP variants, Pro373 and Gln451, might be prothrombotic and contribute to increased risk for VTE.

The findings in this study possess very strong statistical significance. Although the study was limited to males, similar findings are likely to apply to VTE in females.

Because dyslipidemia and dyslipoproteinemia are amenable to pharmacologic treatment, our studies have therapeutic implications for reducing VTE risk. For example, therapy to raise HDL using CETP inhibitors (60-62) would reduce VTE risk.

In summary of this example, venous thromboembolism in males under 55 years old who present with dyslipoproteinemia that includes high density lipoprotein deficiency was associated with the two relatively rare cholesteryl ester transfer protein gene variations, Ala373 to Pro and Arg451 to Gln, which are known to cause decreased levels of high density lipoprotein and elevated levels of CETP mass and activity.

### EXAMPLE3

Antigenic levels of apo Al (the major apolipoprotein of HDL) were measured using the following Study Group.

Study group: The female Caucasian study population consisted of 57 VTE cases and 54 matched controls. The patients had a first thrombosis episode at less than 45 years old and represent a subset from a previously reported study (77). All subjects gave informed consent following institutional guidelines.

Patients under oral anticoagulant and those with known thrombophilic defects, such as antithrombin, protein C, protein S, plasminogen, or heparin cofactor 11 deficiencies, as well as those with APC resistance, factor V Leiden, prothrombin 20210A, or anti-phospholipid antibodies or lupus anticoagulant were excluded as were patients with malignancy, nephrotic syndrome, renal or hepatic dysfunction, inflammatory or infectious disease, or heart failure. For all patients, the diagnosis of VTE was confirmed by objective methods (venography or ultrasonography for VTE of lower limbs, pulmonary angiography or perfusion-ventilation lung scan for pulmonary embolism) at hospital admission.

Statistical analysis: Categorical data between groups were compared using contingency-table analysis (chi-squared test), and continuous data were compared using either unpaired student t-test or Mann-Whitney as appropriate (Prism™ 3.0 software, Graph Pad Software, San Diego, Calif.). All p-values were two-tailed. The multifactorial role of prothrombotic risk factors was assessed using multivariate logistic regression analysis, and adjusted p-values or adjusted odds ratio were calculated using Minitab14 software (Minitab, State College, Pa.).

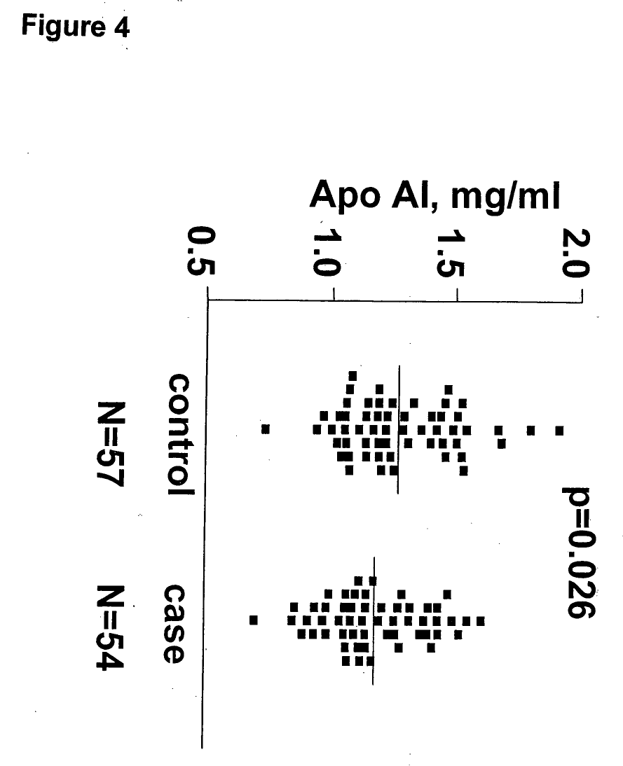

ApoAl levels were lower (p=0.026) in female VTE patients compared to female controls as seen in FIG. 4.

### EXAMPLE 4

For this example, a large cohort of patients who had experienced an episode of venous thrombosis was followed, and the relation between high density lipoprotein parameters and recurrent venous thromboembolism was investigated.

### Methods

Study group: Patients were participants of the Austrian Study on Recurrent Venous Thromboembolism (AUREC), an ongoing, prospective, multicenter cohort study in patients with venous thromboembolism. The characteristics of the study have been reported in detail (36). Between July 1992 and November 2004, 2764 patients older than 18 years, who had been treated with vitamin K-antagonists for at least three months after venous thromboembolism were eligible. Deep vein thrombosis was diagnosed by venography or color duplex sonography (in case of proximal deep vein thrombosis). Pulmonary embolism was diagnosed by ventilation perfusion scan according to the criteria of the Prospective Investigation of Pulmonary Embolism Diagnosis or by multi-slice computed tomography (36). 1992 patients were excluded because of previous thrombosis (329), venous thromboembolism secondary to surgery, trauma or pregnancy (423), antithrombin, protein C, or protein S deficiency (66), the lupus anticoagulant (67), cancer (434), requirement of long-term antithrombotic treatment for reasons other than venous thromboembolism (428), or because material for laboratory testing was not available (122). 26 patients with high factor VIII levels who had participated in an interventional trial investigating the effect of long-term anticoagulation on the risk of recurrence were excluded from the analysis. 97 patients who used statins were also excluded.

The study was approved by the institutional ethics committee and the patients provided written informed consent. Patients entered the study at the time of discontinuation of vitamin K-antagonists and were seen at three-month intervals during the first year and every six months thereafter. They received written information on the symptoms of venous thromboembolism and were instructed to report if symptoms occurred.

Study endpoint: The endpoint of the study was recurrent, symptomatic deep vein thrombosis confirmed by objective methods and by independent clinicians and radiologists.

Blood sampling and laboratory analysis: Venous blood was collected in fasting state after normalization of the prothrombin time (about three weeks after discontinuation of vitamin K-antagonists) into 1/10 volume of 0.11 mmol/L trisodium citrate. Plasma was prepared by centrifugation for 20 minutes at 2000 g and stored at −80° C. Genomic DNA was isolated from blood leukocytes by standard methods. Antithrombin, protein C, protein S, factor V Leiden, prothrombin G20210A mutation, factor VIII, and the lupus anticoagulant were determined as previously reported (36). Plasma levels of apolipoproteins Al and B were measured using immunoturbidometric assay kits (DiaSorin, Stillwater, Minn., USA)(42). Proton nuclear magnetic resonance (NMR) spectroscopy was used to determine levels of 10 lipoprotein subclasses and of lipids in EDTA plasma of 396 patients as described (21,22,42). NMR-derived lipoprotein particle levels are based on the NMR signals that are characteristic of typical lipoprotein particles and are not actual lipid measurements. NMR data are directly proportional to the number of particles, independent of lipid or apolipoprotein per particle which may vary from person to person. NMR spectroscopy was also used to calculate HDL- and LDL-cholesterol levels and shows strong correlation with conventional chemically determined lipid concentration (22). NMR spectroscopy was also used to calculate HDL-cholesterol and LDL-cholesterol levels; this method shows strong correlation with conventional chemical methods for determination of plasma lipids (22). Patients' samples for NMR spectroscopy analysis were selected by recurrence status. For one patient with recurrence, three patients without recurrence were matched according to age and sex.

Statistical analysis: For numerical operations SPSS 12.0 software (SPSS Inc. Headquarters, Chicago, Ill., USA) was used. To test for homogeneity between strata, the log-rank and the generalized Wilcoxon test were applied. Categorical data were checked for homogeneity using contingency-table analyses (by the chi-squared test), and continuous data (presented as mean±SD) were compared using Mann-Whitney U test. Times to recurrence (uncensored observations) or follow up times in patients without recurrence (censored observations) were analyzed according to survival methods. Probability of recurrence was estimated according to Kaplan and Meier (78). Univariate and multivariate Cox proportional hazards models were used to analyze the association between apolipoprotein Al and B levels and the risk of recurrence. Analyses were adjusted for age, sex, body mass index and high factor VIII [dichotomized at the 90^{th} percentile (225 lU/dl)].

For this example, 772 patients with a first spontaneous venous thromboembolism were studied. The mean age of patients was 47±16 years, and 440 of the 772 patients were women (57 percent). Median follow-up of the patients was 48 months. 192 patients left the study because of diagnosis of cancer (15 patients) or of pregnancy (33 patients) or because they required antithrombotic therapy for reasons other than venous thromboembolism (112 patients); 15 patients were lost for follow-up; 17 patients died and for two of them recurrent VTE was the cause of death. The patients were followed until they left the study when data were censored.

Venous thromboembolism recurred in 100 of 772 patients (13 percent). The site for recurrent thrombosis was deep vein thrombosis in 61 patients and pulmonary embolism in 39 patients. According to multivariate analysis, male gender and high factor VIII were major determinants of the risk of recurrence (Table 5). The risk of recurrence increased with increasing body mass index. Heterozygous carriership of either factor V Leiden or factor II G20210A did not confer a significantly higher risk of recurrence as compared with wildtype carriers.

TABLE 5
Relative Risks of Recurrent Venous Thromboembolism
According to Baseline Characteristics

| | Univariate | | Multivariate | |
|---|---|---|---|---|
| | Relative Risk | | Relative Risk | |
| Characteristic | (95% CI) | | (95% CI)* | |
| Age | 1.2 | (1.1-1.4) | 1.1 | (0.9-1.2) |
| (per 10 year increase) | | | | |
| Male sex (vs. female) | 3.0 | (2.0-4.5) | 3.0 | (2.1-4.3) |
| Body mass index | 1.07 | (1.04-1.11) | 1.06 | (1.02-1.09) |
| (per one kg/m² increase) | | | | |
| Duration of anticoagulation | 1.03 | (0.99-1.07) | 1.02 | (0.99-1.06) |
| (per 3 mo increase) | | | | |
| Factor VIII ≧225 IU/dl (vs. | 2.6 | (1.5-4.4) | 1.9 | (1.2-3.1) |
| <225 IU/dl) | | | | |
| Factor V Leiden (vs. absence | 1.2 | (0.9-1.7) | 1.3 | (0.9-1.9) |
| of mutation) | | | | |
| Factor II G20210A (vs. | 1.4 | (0.8-2.4) | 1.4 | (0.8-2.4) |
| absence of mutation) | | | | |

*Multivariate relative risks were adjusted for age, sex, body mass index, duration of anticoagulation, the presence or absence of factor V Leiden, factor II G20210A, and high factor VIII.

Patients with recurrent venous thromboembolism had significantly lower levels of apolipoprotein Al than those without recurrence (1.12±0.22 mg/mL vs. 1.23±0.27 mg/mL, P<0.001). When apolipoprotein Al was entered as a continuous variable in a Cox proportional hazard model, relative risk of recurrence was 0.87 (95 percent confidence interval 0.80 to 0.94) for each 0.1 mg/mL increase in apolipoprotein Al level, and this remained unchanged after adjustment for age, sex, high factor VIII, and body mass index [relative risk 0.88 (95 percent confidence interval 0.80 to 0.97)].

To detect potential linearity in the decrease of risk of recurrence with increasing apolipoprotein Al levels, the relative risk of recurrence among groups of patients corresponding to tertiles of apolipoprotein Al levels was calculated (Table 6). Compared to patients with apolipoprotein Al levels lower than 1.07 mg/mL, relative risk of recurrence was lower [0.78 (95 percent confidence interval 0.50 to 1.22)] among patients with apolipoprotein Al between 1.07 and 1.3 mg/mL and was lowest [0.46 (95 percent confidence interval 0.27 to 0.77)] among those with highest apolipoprotein Al levels (equal or greater than 1.3 mg/mL). Adjustment for potentially confounding variables did not substantially influence these findings.

TABLE 6
Relative Risks of Recurrent Venous Thromboembolism
according to Tertiles of Apolipoprotein AI Levels

| | | | | Univariate | Multivariate |
|---|---|---|---|---|---|
| | | No. of | No. of | Relative Risk | Relative Risk |
| Apolipoprotein AI | | Patients | Recurrences | (95% CI) | (95% CI)* |
| <1.07 | mg/ml | 243 | 43 | 1 | 1 |
| 1.07-1.30 | mg/ml | 267 | 36 | 0.78 (0.50-1.22) | 0.76 (0.48-1.21) |
| ≧1.30 | mg/ml | 262 | 21 | 0.46 (0.27-0.77) | 0.50 (0.28-0.88) |

*adjusted for age, sex, body mass index, high factor VIII

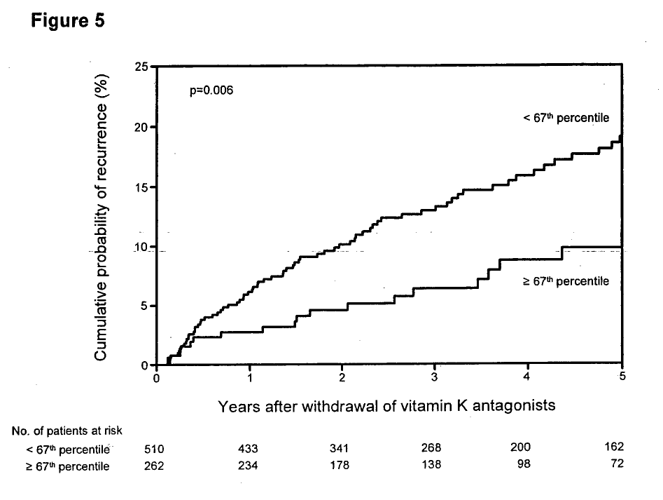

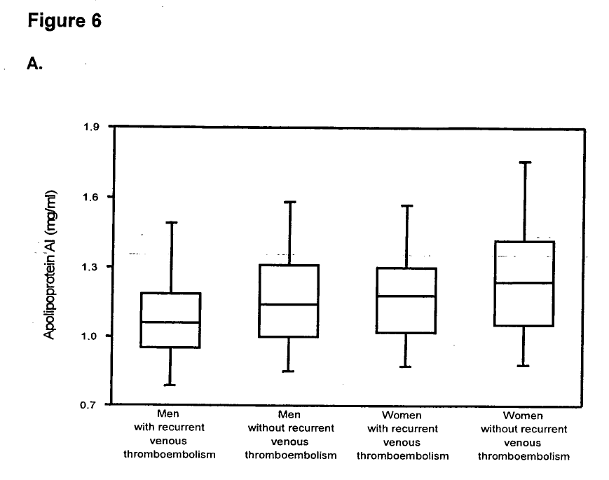

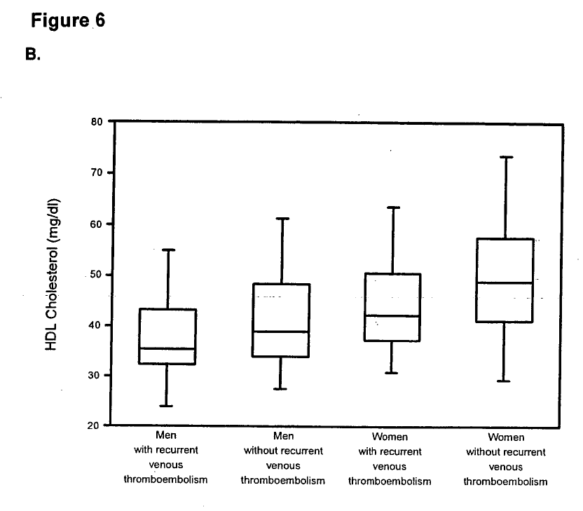

Patients were dichotomized into those with apolipoprotein Al levels equal to or greater than 1.3 mg/mL (corresponding to the 67^{th} percentile of thrombosis patients) and in those with lower levels. Table 7 shows the characteristics of these patients. Patients with lower apolipoprotein Al were younger and had a higher body mass index. According to Kaplan-Meier analysis, a clear divergence between the rates of recurrence between these two groups was seen (FIG. 5). At four years, the cumulative probability of recurrence was 8.8 percent (95 percent confidence interval 4.6 to 12.9 percent) among patients with apolipoprotein Al levels equal to or greater than 1.3 mg/mL, as compared with 15.9 percent (95 percent confidence interval 12.2 to 19.5 percent), among patients with lower apolipoprotein Al levels (P=0.006). According to univariate analysis, high apolipoprotein Al conferred a relative risk of recurrence of 0.51 (95 percent confidence interval 0.32 to 0.83). After adjustment for age, sex, high factor VIII, and body mass index, the risk of recurrence was 0.58 (95 percent confidence interval 0.35 to 0.97), which translates into a 43 percent lower relative risk of recurrence as compared with lower apolipoprotein Al levels.

TABLE 7
Baseline Characteristics of the 772 Patients According to
Apolipoprotein AI Level above or below the 67^{th} percentile.

| | <67^{th} | | |
|---|---|---|---|
| Characteristics | percentile | ≧67^{th} percentile | P |
| Age at first VTE (yrs) | 45 ± 16 | 51 ± 16 | <0.001 |
| Men, no. (%) | 250 (49) | 82 (31) | <0.001 |
| Duration of anticoagulation - | 8 ± 9 | 8 ± 6 | 1.0 |
| mo | | | |
| Body mass index (kg/m²) | 27 ± 5 | 26 ± 4 | <0.001 |
| Factor V Leiden, no. (%) | 149 (29) | 71 (27) | 0.6 |
| Factor II G20210A, no. (%) | 33 (7) | 23 (9) | 0.2 |
| Factor VIII (IU/dl) | 163 ± 47 | 170 ± 49 | 0.05 |

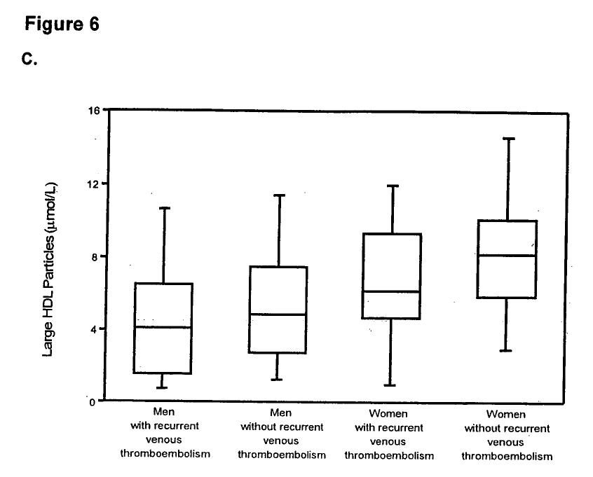

Apolipoprotein Al levels were significantly lower among men as compared with women (1.15±0.23 vs. 1.26±0.28 mg/mL, P<0.001). The proportion of men in the lower tertile of apolipoprotein Al levels (less than 1.07 mg/mL) was higher as compared with that of women [126 of 332 (38 percent) vs. 117 of 440 (27 percent, P<0.001]. Conversely, women were more prevalent among patients with apolipoprotein Al levels in the upper tertile (equal or greater than 1.3 mg/mL) [180 of 440 (41 percent) vs. 82 of 332 (25 percent), P<0.001)]. Men with recurrence had lowest apolipoprotein Al levels, while women without recurrence had highest levels (FIG. 6, panel A). As well known to those skilled in the art, women intrinsically had higher levels of HDL than men based on each HDL parameter.

High density lipoprotein cholesterol levels were determined using NMR spectroscopy analysis of plasmas for 261 men and 132 women. A positive correlation between apolipoprotein Al and high density lipoprotein cholesterol levels was found (r²=0.87, P<0.001). High density lipoprotein cholesterol levels were lowest among men with recurrence and were highest among women without recurrence (FIG. 6, panel B).

Large HDL particle concentrations in subjects' plasma samples were determined using NMR spectroscopy. As seen in FIG. 6C, men with recurrent venous thrombosis had lower levels of these particles than men without recurrence, and the same was true for women with recurrence compared to women without recurrence.

### EXAMPLE 5

In this example, male VTE subjects were compared with matched male controls to determine the characteristics of differences in HDL and lipoprotein parameters. For this purpose, the Study Group was a subset of young adult males taken from the cohorts described in Example 1. None of these subjects was taking lipid-lowering medications.

### Methods

Study group: In this example, male VTE patients (n=49) and age matched controls (n=49) were analyzed for lipid and lipoprotein characterization. Clinical characteristics, the frequency of identified risk factors and serum lipid data are shown in Table 8. Forty of 49 VTE patients (82%) presented with idiopathic VTE, defined as events that did not occur within 90 days after surgery, trauma, or major immobilization. Clinical conditions which are associated with changes in lipid metabolism were recorded for VTE patients and controls. Diabetes was present in one VTE patient. Hypertension was present in 3 VTE patients and was not present in any controls. Prior smoking history was similar in male VTE patients compared with controls (8 vs. 6, p=0.79). Current smoking was similar in male VTE patients compared with male controls (5 vs. 4, p=1.0). Of the VTE patients, 18 (37%) had experienced more than one episode of thrombosis and 17 (35%) had documented pulmonary embolism. Eighty-four percent of patients were taking warfarin when blood was donated. Cancer was not known to be present in VTE patients and controls.

TABLE 8
Study population

| | Control | VTE | |
|---|---|---|---|
| Variables | N = 49 | N = 49 | p value |
| Age, yr (SD) | 46.3 (8.2) | 46.7 (8.6) | |
| Ethnic Group, % | | | |
| Non-Hispanic White | 91.8 | 91.8 | |
| Body-mass Index, kg m⁻² | 27.2 (4.3) | 30.7 (5.9) | 0.003** |
| (SD) | | | |
| Risk factors, number of | | | |
| subjects (%) | | | |
| Family History for VTE | 4 (8.1) | 18 (37) | 0.0001** |
| Factor V Leiden | 2 (4.1) | 13 (26.5) | 0.006** |
| Prothrombin 20210A | 2 (4.1) | 7 (14.3) | 0.13 |
| Idiopathic | | 40 (81.6) | |
| Clinical Lipids, mean (SD) | | | |
| total cholesterol, mg/dl | 205 (41) | 215 (44) | 0.30 |
| Triglyceride, mg/dl | 143 (84) | 159 (98) | 0.44 |
| HDL-C, mg/dl | 53.6 (15.0) | 46.3 (17.8) | 0.03* |
| LDL-C, mg/dl | 122 (33) | 135 (41) | 0.11 |
| LDL-C/HDL-C ratio | 2.45 (0.91) | 3.17 (1.24) | 0.001** |

*indicates 0.01 ≦ p < 0.05; and
**indicates 0.001 ≦ p < 0.01.

The methods for blood collection, lipid analysis, apolipoprotein assays, NMR analysis and DNA analysis were as indicated in Example 1

Statistical analysis: Cases were matched in a 1:1 ratio to controls by the following factors: age (±2 years), gender and ethnicity. The differences between lipoprotein parameters of cases and matched controls were calculated and tested against the null hypothesis of a difference of 0 (no difference) with the paired t-test or Wilcoxon rank test (Prizm™ 3.0 software, GraphPad Software, San Diego, Calif.). All p values were two-tailed. McNemar's test was used to evaluate the difference in proportion between matched pairs for categorical variables (e.g. VTE positive family history, smoking status, etc).

Lipid levels were analyzed as categorical variables after division into quartiles, with either the lowest or highest quartile used as the reference category. Conditional logistic regression (accounting for age and sex matching) was used to estimate odds ratios (ORs). Adjustments for well known risk factors BMI, factor V Leiden and prothrombin 20210A were also performed using conditional logistic regression (STATA 8.0, Stata Corporation, College Station, Tex.). Individual models were used to calculate the adjusted OR for each lipid parameter. P values for linear trend across quartiles of each biomarker were calculated without adjustment for multiple comparisons. To evaluate the association of VTE with genetic polymorphisms, both conditional logistic regression and chi-squared analysis were used for comparison of genotype and allele frequency between the two groups.

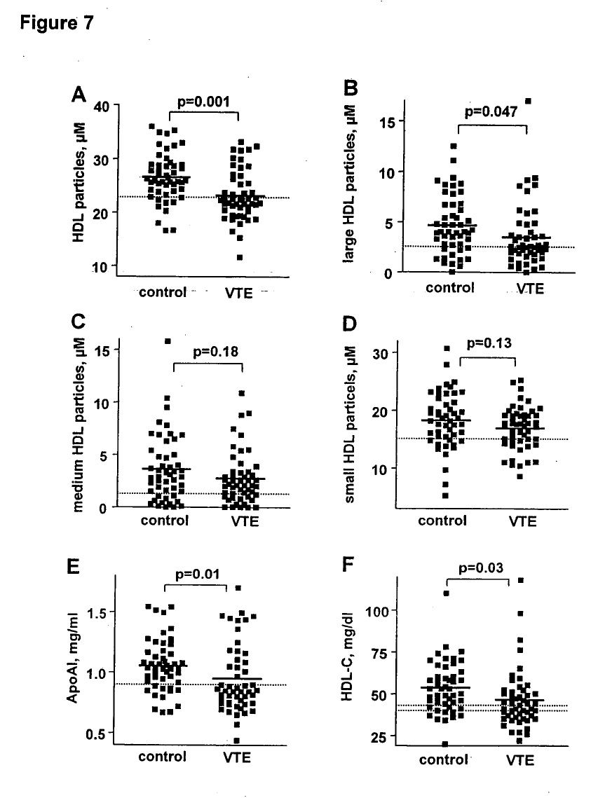

Male VTE cases had a significantly lower mean HDL particle concentration than controls (p=0.001) (FIG. 7A). Among HDL lipoprotein subclasses, large HDL particle concentrations were lower in patients than controls (p=0.047), whereas medium and small HDL particles were not significantly different (FIGS. 7B-D). The HDL mean particle size was also smaller in VTE cases than controls (p=0.04).

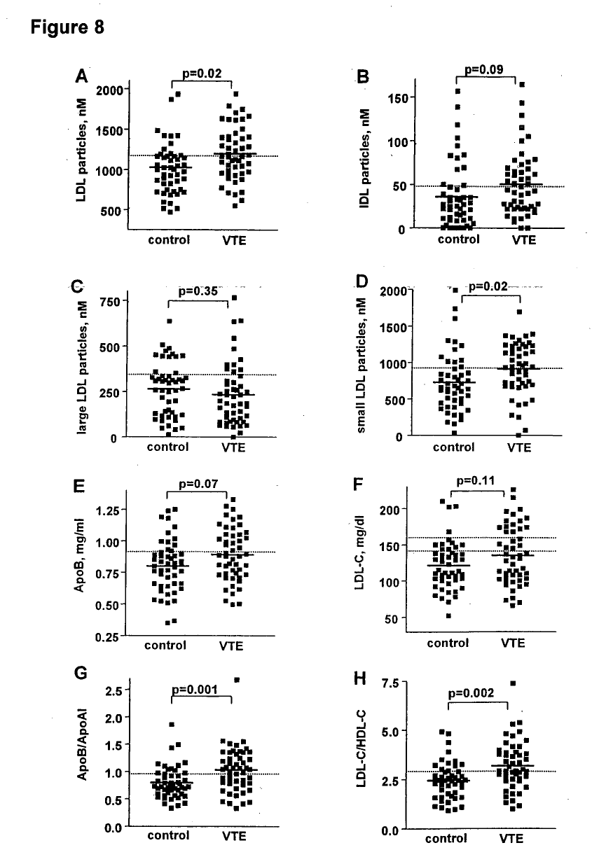

LDL lipoprotein particle concentrations in VTE patients were significantly higher than in controls (FIG. 8A, p=0.02). Among LDL lipoprotein subclasses small LDL particle concentrations were higher in cases (p=0.02), whereas IDL and large LDL particle levels showed no statistical difference (FIGS. 8B-D). The two subgroups of small LDL particles, namely medium small particles and very small LDL particles, were elevated in VTE cases (p=0.02 and 0.03, respectively, data not shown). The LDL mean particle size was smaller in cases compared to controls (p=0.04). No statistically significant differences between cases and controls were observed for VLDL particle total concentration or for VLDL subclasses (large, medium and small VLDL particles) or for VLDL particle size.

Immunoassay data for apolipoproteins Al and B were consistent with NMR-based lipoprotein subclass data. ApoAl levels were lower in VTE patients (p=0.01) (FIG. 7E) although there was no difference in apoB levels (FIG. 8E) in VTE patients compared to controls. The apoB to apoAl ratios for VTE patients were significantly higher than for controls (p=0.002) (FIG. 8G), and the apoB/apoAl mean ratio difference was statistically stronger than the difference in mean values for either ApoAl or ApoB alone.

For VTE patients compared to controls, HDL-cholesterol (HDL-C) was lower (p=0.03) whereas there was no difference in LDL-cholesterol (LDL-C) (p=0.11) (FIGS. 7F and 8F). Remarkably, however, the LDL-C to HDL-C mean ratio was significantly higher for VTE patients compared to controls (p=0.001) (FIG. 8H).

Quartile-based odds ratios (OR) for association with VTE with lipoprotein variables were calculated by comparing quartile 1 (lowest HDL) with quartiles 2-4 or quartile 4 (highest LDL) with quartiles 1-3 for reduced HDL parameters or elevated LDL parameters, respectively (Table 9). Low levels of total HDL particle concentration and of large HDL particle concentration were associated with increased VTE risk with an OR=6.5 (95% Cl: 2.3-19) and 2.8 (95% Cl: 1.2-6.2), respectively. Smaller HDL mean particle size had a significant OR of 3.2 (95% Cl: 1.3-7.9). High levels (>75% of controls) of total LDL particle concentration and of two subfractions, IDL and small LDL particle concentrations, were significantly associated with the risk of VTE with an OR=2.2 (95% Cl: 1.04.9), 2.7 (95% Cl: 1.0-6.8) and 3.1 (95% Cl: 1.3-7.4), respectively. Two subpopulations of the small LDL particle subclass, i.e., medium small and very small LDL particles, gave an OR of 3.0 and 3.1 (95% Cl: 1.3-7.0 and 1.3-7.4), respectively, (data not shown), a value similar to the OR of 3.1 for small LDL particle concentration (Table 9). After adjustment for the known VTE risk factors, factor V Leiden, prothrombin 20210A, and BMI, all statistically significant quartile-based OR values retained statistical significance, with the exception of the concentration of large HDL particles (p=0.056) (Table 9). The reduction in LDL mean particle size had a significant OR of 7.6 (95% Cl: 1.5-39) after adjustment for factor V Leiden, prothrombin 20210A and BMI.

Plasma levels of apoAl and apoB were determined by standard immunoassays. For VTE, based on quartile analyses, low levels of apoAl gave significant OR values, OR=6.0 (95% Cl: 2.1-17), as did the ratio of ApoB/ApoAl, OR=6.3 (95% Cl: 1.9-21). After adjustment for factor V Leiden, prothrombin 20210A and BMI, the significance remained for these ORs (Table 6). Based on clinical lab serum cholesterol data, the OR for low HDL-C was statistically significant [OR=3.0 (95% Cl: 1.3-7.1)] while that for elevated LDL-C did not quite achieve statistical significance [OR=1.9 (95% Cl: 0.84-4.2)]. After adjustment for factor V Leiden, prothrombin 20210A, and BMI, the OR for elevated LDL-C achieved statistical significance [OR=3.9 (95% Cl: 1.1-14)], and the OR for low HDL-C was not quite statistically significant [OR=2.6 (95% Cl: 0.78-8.9)]. For the OR for VTE, a ratio of LDL-C/HDL-C in the upper quartile was statistically significant for increased VTE risk [OR=2.7 (95% Cl: 1.1-6.5) without adjustment, and the OR was 5.0 (95% Cl: 1.3-20) after adjustment] (Table 10). For LDL-C levels above 160 mg/dl, the OR for VTE was 3.5 (95% Cl: 1.2-11) while for HDL-C below 40 mg/dl, the OR for VTE was 2.8 (95% Cl: 1.1-7.2) (data not shown).

TABLE 9
Odds ratios for VTE associated with LDL-related and HDL-related parameters.

| | unadjusted values | | | adjusted values | | |
|---|---|---|---|---|---|---|
| Variables | OR | (95% CI) | p value | OR | (95% CI) | p value |
| HDL-associated variables | | | | | | |
| HDL concentration | 6.5 | (2.3-19) | <0.001 *** | 4.4 | (1.2-16) | 0.03 * |
| large HDL | 2.8 | (1.2-6.2) | 0.01 * | 2.9 | (0.97-8.9) | 0.056 |
| medium HDL | 1.3 | (0.49-3.2) | 0.64 | 1.0 | (0.27-3.8) | 0.98 |
| small HDL | 1.8 | (0.68-5.0) | 0.23 | 1.7 | (0.46-6.4) | 0.42 |
| HDL size | 3.2 | (1.3-7.9) | 0.01 * | 4.8 | (1.1-20) | 0.03 * |
| ApoAl | 6.0 | (2.1-17) | <0.001 *** | 4.7 | (1.1-21) | 0.04 * |
| HDL-C | 3.0 | (1.3-7.1) | 0.01 * | 2.6 | (0.78-8.9) | 0.12 |
| LDL-associated variables | | | | | | |
| LDL concentration | 2.2 | (1.0-4.9) | 0.047 * | 3.6 | (1.0-1.6) | 0.04 * |
| IDL | 2.7 | (1.0-6.8) | 0.04 * | 1.3 | (1.1-1.5) | 0.0097 ** |
| large LDL | 0.9 | (0.37-2.2) | 0.82 | 0.46 | (0.11-2.0) | 0.29 |
| small LDL | 3.1 | (1.3-7.4) | 0.008 ** | 9.7 | (1.9-50) | 0.007 ** |
| LDL size | 2.0 | (0.90-4.5) | 0.09 | 7.6 | (1.5-39) | 0.02 * |
| ApoB | 2.1 | (0.91-4.9) | 0.08 | 1.9 | (0.63-5.8) | 0.25 |
| LDL-C | 1.9 | (0.84-4.2) | 0.12 | 3.9 | (1.1-14) | 0.04 * |
| Ratio | | | | | | |
| LDL-C/HDL-C | 2.7 | (1.1-6.5) | 0.02 * | 5.0 | (1.3-20) | 0.02 * |
| ApoB/ApoAl | 6.3 | (1.9-21) | 0.003 ** | 6.1 | (1.2-30) | 0.03 * |

The quartile-based odds ratios (OR) for VTE with the levels of HDL-associated parameters below the 25%-ile of controls or with the levels of LDL-associated parameters above the 75%-ile of controls are shown based on conditional logistic regression analysis. OR values adjusted for factor V Leiden, prothrombin 20210A, and BMI are also shown. 95% CI denotes 95% confidential interval.
* indicates 0.01 ≦ p < 0.05;
** indicates 0.001 ≦ p < 0.01; and
*** indicates p < 0.001.

To evaluate the linear association of VTE risk with lipid parameter levels, matched pair analysis of quartiles was made and the p values for a trend were calculated (Table 10). Lower levels of total HDL particle concentration, large HDL particle concentration and higher levels of total LDL, IDL and small LDL particle concentrations were associated with increased VTE risk (p=0.004, 0.009, 0.03, 0.03 and 0.03, respectively). Smaller HDL size and larger LDL size were also associated with increased VTE risk (p=0.02 and 0.01, respectively).

TABLE 10
Trend analysis for association of VTE with HDL and LDL
parameters based on quartiles.

| | |
|---|---|
| Variables | p trend |

HDL-associated variables

| | |
|---|---|
| HDL concentration | 0.004** |
| large HDL | 0.009** |
| medium HDL | 0.15 |
| small HDL | 0.07 |
| HDL size | 0.02* |
| ApoAI | 0.02* |
| HDL-C | 0.005** |

LDL-associated variables

| | |
|---|---|
| LDL concentration | 0.03* |
| IDL | 0.03* |
| large LDL | 0.19 |
| small LDL | 0.03* |
| LDL size | 0.01* |
| ApoB | 0.18 |
| LDL-C | 0.35 |
| Ratio | |
| LDL-C/HDL-C | 0.01* |
| ApoB/ApoAI | 0.002** |

*indicates 0.01 ≦ p < 0.05;
**indicates 0.001 ≦ p < 0.01; and
***indicates p < 0.001.

To identify genetic influences contributing to dyslipoproteinemia in VTE patients, SNPs in three key genes that influence the spectrum of lipoprotein subpopulations and HDL-C levels, hepatic lipase (LIPC -514C/T), endothelial lipase (LIPG T111I) and CETP (TaqI B and 1405V), were determined. Using chi-squared analysis for difference in allele frequency between VTE cases and controls, the CETP TaqI B2 allele was significantly less common in VTE cases than controls (p=0.04) (Table 11). No difference in allelic frequency was found for the studied SNPs of hepatic lipase or endothelial lipase. To evaluate the association of genotype with VTE, conditional logistic regression was also performed. The CETP TaqI B genotype was significantly associated with VTE (p=0.017), whereas no association was observed for CETP 1405V, LIPC or LIPG polymorphisms (p=0.15, 0.60 and 0.48, respectively) in the study group of this example.

TABLE 11
Allele frequencies of the less common allele for single
nucleotide polymorphisms in hepatic lipase (LIPC),
endothelial lipase (LIPG) and cholesteryl ester transfer
protein (CETP) in VTE cases and controls.

| | Controls | VTE | |
|---|---|---|---|
| | n = 49 | n = 49 | p |
| LIPC-514C/T | 0.22 | 0.25 | 0.67 |
| LIPG T111I | 0.33 | 0.28 | 0.44 |
| CETP TaqI B1/B2 | 0.47 | 0.33 | 0.04 |
| CETP I405V | 0.31 | 0.20 | 0.10 |

### REFERENCES

(1) Rosendaal F R. Venous thrombosis: a multicausal disease. Lancet 1999;353:1167-73.

(2) Lensing A W, Prandoni P, Prins M H, Buller H R. Deep-vein thrombosis. Lancet 1999;353:479-85.

(3) Bertina R M, Koeleman B P, Koster T, Rosendaal F R, Dirven R J, de Ronde H et al. Mutation in blood coagulation factor V associated with resistance to activated protein C. Nature 1994;369:64-7.

(4) Greengard J S, Sun X, Xu X, Fernandez J A, Griffin J H, Evatt B. Activated protein C resistance caused by Arg506Gln mutation in factor Va. Lancet 1994;343:1361-2.

(5) Goodnight S H, Griffin J H. Hereditary Thrombophilia. In: Beutler E, Lichtman M A, Coller B A, Kipps T J, Seligsohn U, editors. 6th ed. Williams Hematology. New York: McGraw-Hill, 2001: 1697-1714.

(6) Prandoni P, Bilora F, Marchiori A, Bernardi E, Petrobelli F, Lensing A W et al. An association between atherosclerosis and venous thrombosis. N Engl J Med 2003;348:1435-41.

(7) Grady D, Wenger N K, Herrington D, Khan S, Furberg C, Hunninghake D et al. Postmenopausal hormone therapy increases risk for venous thromboembolic disease. The Heart and Estrogen/progestin Replacement Study. Ann Intern Med 2000;132:689-96.

(8) Herrington D M, Vittinghoff E, Lin F, Fong J, Harris F, Hunninghake D et al. Statin therapy, cardiovascular events, and total mortality in the Heart and Estrogen/Progestin Replacement Study (HERS). Circulation 2002; 105:2962-7.

(9) Ray J G, Mamdani M, Tsuyuki R T, Anderson D R, Yeo E L, Laupacis A. Use of statins and the subsequent development of deep vein thrombosis. Arch Intern Med 2001;161:1405-10.

(10) Deguchi H, Fernandez J A, Pabinger I, Heit J A, Griffin J H. Plasma glucosylceramide deficiency as potential risk factor for venous thrombosis and modulator of anticoagulant protein C pathway. Blood 2001 ;97:1907-14.

(11) Deguchi H, Fernandez J A, Griffin J H. Neutral glycosphingolipid-dependent inactivation of coagulation factor Va by activated protein C and protein S. J Biol Chem 2002;277:8861-5.

(12) Griffin J H, Kojima K, Banka C L, Curtiss L K, Fernandez J A. High-density lipoprotein enhancement of anticoagulant activities of plasma protein S and activated protein C. J Clin Invest 1999;103:219-27.

(13) Griffin J H, Fernandez J A, Deguchi H. Plasma lipoproteins, hemostasis and thrombosis. Thromb Haemost 2001 ;86:386-94.

(14) Otvos J D, Jeyarajah E J, Cromwell W C. Measurement issues related to lipoprotein heterogeneity. Am J Cardiol 2002;90:22i -29i.

(15) Otvos J D, Jeyarajah E J, Bennett D W, Krauss R M. Development of a proton nuclear magnetic resonance spectroscopic method for determining plasma lipoprotein concentrations and subspecies distributions from a single, rapid measurement. Clin Chem 1992;38:1632-8.

(16) Ordovas J M, Cupples L A, Corella D, Otvos J D, Osgood D, Martinez A et al. Association of cholesteryl ester transfer protein-TaqIB polymorphism with variations in lipoprotein subclasses and coronary heart disease risk: the Framingham study. Arterioscler Thromb Vase Biol 2000;20:1323-9.

(17) Guerra R, Wang J, Grundy S M, Cohen J C. A hepatic lipase (LIPC) allele associated with high plasma concentrations of high density lipoprotein cholesterol. Proc Natl Acad Sci USA 1997;94:4532-7.

(18) Grundy S M, Vega G L, Otvos J D, Rainwater D L, Cohen J C. Hepatic lipase activity influences high density lipoprotein subclass distribution in normotriglyceridemic men. Genetic and pharmacological evidence. J Lipid Res 1999;40:229-34.

(19) Barzilai N, Atzmon G, Schechter C, Schaefer E J, Cupples A L, Lipton R et al. Unique lipoprotein phenotype and genotype associated with exceptional longevity. JAMA 2003;290:2030-40.

(20) Ma K, Cilingiroglu M, Otvos J D, Ballantyne C M, Marian A J, Chan L. Endothelial lipase is a major genetic determinant for high-density lipoprotein concentration, structure, and metabolism. Proc Natl Acad Sci USA 2003; 100:2748-53.

(21) Otvos J D. Measurement of lipoprotein subclass profiles by nuclear magnetic resonance spectroscopy. Clin Lab 2002;48:171-80.

(22) Freedman D S, Otvos J D, Jeyarajah E J, Shalaurova I, Cupples L A, Parise H et al. Sex and Age Differences in Lipoprotein Subclasses Measured by Nuclear Magnetic Resonance Spectroscopy: The Framingham Study. Clin Chem 2004;50:1189-200.

(23) Xu X, Bauer K A, Griffin J H. Two multiplex PCR-based DNA assays for the thrombosis risk factors prothrombin G20210A and coagulation factor V G1691A polymorphisms. Thromb Res 1999;93:265-9.

(24) Kakko S, Tamminen M, Paivansalo M, Karma H, Rantala A O, Lilja M et al. Cholesteryl ester transfer protein gene polymorphisms are associated with carotid atherosclerosis in men. Eur J Clin Invest 2000;30:18-25.

(25) Brousseau M E, O'Connor J J, Jr., Ordovas J M, Collins D, Otvos J D, Massov T et al. Cholesteryl ester transfer protein TaqI B2B2 genotype is associated with higher HDL cholesterol levels and lower risk of coronary heart disease end points in men with HDL deficiency: Veterans Affairs HDL Cholesterol Intervention Trial. Arterioscler Thromb Vasc Biol 2002;22:1148-54.

(26) Walldius G, Jungner I, Holme I, Aastveit A H, Kolar W, Steiner E. High apolipoprotein B, low apolipoprotein A-I, and improvement in the prediction of fatal myocardial infarction (AMORIS study): a prospective study. Lancet 2001 ;358:2026-33.

(27) Gordon D J, Probstfield J L, Garrison R J, Neaton J D, Castelli W P, Knoke J D et al. High-density lipoprotein cholesterol and cardiovascular disease. Four prospective American studies. Circulation 1989;79:8-15.

(28) Gordon T, Castelli W P, Hjortland M C, Kannel W B, Dawber T R. High density lipoprotein as a protective factor against coronary heart disease. The Framingham Study. Am J Med 1977;62:707-14.

(29) Maciejko J J, Holmes D R, Kottke B A, Zinsmeister A R, Dinh D M, Mao S J. Apolipoprotein A-I as a marker of angiographically assessed coronary-artery disease. N Engl J Med 1983;309:385-9.

(30) Rhoads G G, Gulbrandsen C L, Kagan A. Serum lipoproteins and coronary heart disease in a population study of Hawaii Japanese men. N Engl J Med 1976;294:293-8.

(31) Stampfer M J, Sacks F M, Salvini S, Willett W C, Hennekens C H. A prospective study of cholesterol, apolipoproteins, and the risk of myocardial infarction. N Engl J Med 1991;325:373-81.

(32) Couture P, Otvos J D, Cupples L A, Lahoz C, Wilson P W, Schaefer E J et al. Association of the C-514T polymorphism in the hepatic lipase gene with variations in lipoprotein subclass profiles: The Framingham Offspring Study. Arterioscler Thromb Vasc Biol 2000;20:815-22.

(33) Barter P J, Brewer H B, Jr., Chapman M J, Hennekens C H, Rader D J, Tall A R. Cholesteryl ester transfer protein: a novel target for raising HDL and inhibiting atherosclerosis. Arterioscler Thromb Vasc Biol 2003;23:160-7.

(34) Brousseau M E, Schaefer E J, Wolfe M L, Bloedon L T, Digenio A G, Clark R W et al. Effects-of an inhibitor of cholesteryl ester transfer protein on HDL cholesterol. N Engl J Med 2004;350:1505-15.

(35) Klerkx A H, Tanck M W, Kastelein J J, Molhuizen H O, Jukema J W, Zwinderman A H et al. Haplotype analysis of the CETP gene: not TaqIB, but the closely linked-629C→A polymorphism and a novel promoter variant are independently associated with CETP concentration. Hum Mol Genet 2003; 12:111-23.

(36) Kyrle P A, Minar E, Bialonczyk C, Hirschl M, Weltermann A, Eichinger S. The risk of recurrent venous thromboembolism in men and women. N Engl J Med 2004;350:2558-63.

(37) Tsai A W, Cushman M, Rosamond W D, Heckbert S R, Polak J F, Folsom A R. Cardiovascular risk factors and venous thromboembolism incidence: the longitudinal investigation of thromboembolism etiology. Arch Intern Med 2002;162:1182-9.

(38) Kyrle P A, Eichinger S. Deep vein thrombosis. Lancet. 2005; 365: 1163-1174.

(39) Rosendaal F R. Venous thrombosis: a multicausal disease. Lancet. 1999;353:1167-1173.

(40) Kyrle P A, Minar E, Bialonczyk C, Hirschl M, Weltermann A, Eichinger S. The risk of recurrent venous thromboembolism in men and women. N Engl J Med. 2004; 350:2558-2563.

(41) Baglin T, Luddington R, Brown K, Baglin C. High risk of recurrent venous thromboembolism in men. J Thromb Haemost. 2004;2:2152-2155.

(42) Deguchi H, Pecheniuk N M, Elias D J, Averell P M, Griffin J H. High-density lipoprotein deficiency and dyslipoproteinemia associated with venous thrombosis in men. Circulation. 2005; 112:893-899.

(43) Prandoni P, Bilora F, Marchiori A, Bernardi E, Petrobelli F, Lensing A W, Prins M H, Girolami A. An association between atherosclerosis and venous thrombosis. N Engl J Med. 2003;348:1435-1441.

(44) Wilson P W, D'Agostino R B, Levy D, Belanger A M, Silbershatz H, Kannel W B. Prediction of coronary heart disease using risk factor categories. Circulation. 1998;97: 1837-1847.

(45) Grundy S M. Primary prevention of coronary heart disease: integrating risk assessment with intervention. Circulation. 1999; 100:988-998.

(46) Fuster V, Gotto A M, Jr. Risk reduction. Circulation.2000;102:1V94-IV102.

(47) Griffin J H, Fernandez J A, Deguchi H. Plasma lipoproteins, hemostasis and thrombosis. Thromb Haemost. 2001;86:386-394.

(48) Deguchi H, Fernandez J A, Pabinger I, Heit J A, Griffin J H. Plasma glucosylceramide deficiency as potential risk factor for venous thrombosis and modulator of anticoagulant protein C pathway. Blood. 2001;97:1907-1914.

(49) Deguchi H, Fernandez J A, Griffin J H. Neutral glycosphingolipid-dependent inactivation of coagulation factor Va by activated protein C and protein S. J Biol Chem. 2002;277:8861-8865.

(50) Griffin J H, Kojima K, Banka C L, Curtiss L K, Fernandez J A. High-density lipoprotein enhancement of anticoagulant activities of plasma protein S and activated protein C. J Clin Invest. 1999;103:219-227.

(51) Deguchi H, Yegneswaran S, Griffin J H. Sphingolipids as bioactive regulators of thrombin generation. J Biol Chem. 2004;279:12036-12042.

(52) Agerholm-Larsen B, Tybjaerg-Hansen A, Schnohr P, Steffensen R, Nordestgaard B G. Common cholesteryl ester transfer protein mutations, decreased HDL cholesterol, and possible decreased risk of ischemic heart disease. Circulation. 2000; 102:2197-2203.

(53) Boekholdt S M, Thompson J F. Natural genetic variation as a tool in understanding the role of CETP in lipid levels and disease. J Lipid Res. 2003;44: 1080-1093.

(54) Barter P J, Brewer H B Jr, Chapman M J, Hennekens C H, Rader D J, Tall A R. Cholesteryl ester transfer protein: a novel target for raising HDL and inhibiting atherosclerosis. Arterioscler Thromb Vasc Biol. 2003;23: 160-167.

(55) Brewer H B Jr. High-density lipoproteins: a new potential therapeutic target for the prevention of cardiovascular disease. Arterioscler Thromb Vasc Biol. 2004 24:387-391.

(56) Kakko S, Tamminen M, Paivansalo M, Kauma H, Rantala A O, Lilja M, Reunanen A, Kesaniemi Y A, Savolainen M J. Variation at the cholesteryl ester transfer protein gene in relation to plasma high density lipoproteins cholesterol levels and carotid intima-media thickness. Eur J Clin Invest. 2000; 30:18-25.

(57) Lloyd D B, Lira M E, Wood L S, Durham L K, Freeman T B, Preston G M, Qiu X, Sugarman E, Bonnette P, Lanzetti A, Milos P M, Thompson J F. Cholesteryl ester transfer protein variants have differential stability but uniform inhibition by Torcetrapib. J Biol Chem. 2005; 280:14918-14922.

(58) Otvos J D. Measurement of lipoprotein subclass profiles by nuclear magnetic resonance spectroscopy. In: Rifai N, Warnick G R, Dominiczak M H, editors. Handbook of Lipoprotein Testing. Washington D.C.: American Association for Clincal Chemistry Press, 1997: 497-508.

(59) Freedman D S, Otvos J D, Jeyarajah E J, Shalaurova I, Cupples L A, Parise H, D'Agostino R B, Wilson P W, Schaefer E J. Sex and Age Differences in Lipoprotein Subclasses Measured by Nuclear Magnetic Resonance Spectroscopy: The Framingham Study. Clin Chem. 2004;50:1189-1200.

(60) Brousseau M E, Schaefer E J, Wolfe M L, Bloedon L T, Digenio A G, Clark R W, Mancuso J P, Rader D J. Effects of an inhibitor of cholesteryl ester transfer protein on HDL cholesterol. N Engl J Med. 2004;350:1505-1515.

(61) Clark R W, Sutfin T A, Ruggeri R B, Willauer A T, Sugarman E D, Magnus-Aryitey G, Cosgrove P G, Sand T M, Wester R T, Williams J A, Perlman M E, Bamberger M J. Raising high-density lipoprotein in humans through inhibition of cholesteryl ester transfer protein: an initial multidose study of torcetrapib. Arterioscler Thromb Vasc Biol. 2004;24:490-497.

(62) Brousseau M E, Diffenderfer M R, Millar J S, Nartsupha C, Asztalos B F, Welty F K, Wolfe M L, Rudling M, Bjorkhem I, Angelin B, Mancuso J P, Digenio A G, Rader D J, Schaefer E J. Effects of cholesteryl ester transfer protein inhibition on high-density lipoprotein subspecies, apolipoprotein A-I metabolism, and fecal sterol excretion. Arterioscler Thromb Vasc Biol. 2005;25: 1057-1064.

(63) Nofer J R, Levkau B, Wolinska I, Junker R, Fobker M, von Eckardstein A, Seedorf U, Assmann G. Suppression of endothelial cell apoptosis by high density lipoproteins (HDL) and HDL-associated lysosphingolipids. J Biol Chem. 2001 ;276:34480-34485.

(64) Mineo C, Shaul P W. HDL stimulation of endothelial nitric oxide synthase: a novel mechanism of HDL action. Trends Cardiovasc Med. 2003; 13:226-231.

(65) Barter P J, Nicholls S, Rye K A, Anantharamaiah G M, Navab M, Fogelman A M. Anti-inflammatory properties of HDL. Circ Res. 2004;95:764-772.

(66) von Eckardstein A, Hersberger M, Rohrer L. Current understanding of the metabolism and biological actions of HDL. Curr Opin Clin Nutr Metab Care. 2005;8:147-152.

(67) Kawasaki T, Kambayashi J, Ariyoshi H, Sakon M, Suehisa E, Monden M. Hypercholesterolemia as a risk factor for deep-vein thrombosis. Thromb Res. 1997;88:67-73.

(68) Lippi G, Brocco G, Manzato F, Guidi G. Relationship between venous thromboembolism and lipid or lipoprotein disorders. Thromb Res. 1999;95:353-354.

(69) Tsai A W, Cushman M, Rosamond W D, Heckbert S R, Polak J F, Folsom A R. Cardiovascular risk factors and venous thromboembolism incidence: the longitudinal investigation of thromboembolism etiology. Arch Intern Med. 2002;162:1182-1189.

(70) Gonzalez-Ordonez A J, Fernandez-Carreira J M, Fernandez-Alvarez C R, Venta O R, Macias-Robles M D, Gonzalez-Franco A, Arias Garcia M A. The concentrations of soluble vascular cell adhesion molecule-1 and lipids are independently associated with venous thromboembolism. Haematologica. 2003;88:1035-1043.

(71) Doggen C J, Smith N L, Lemaitre R N, Heckbert S R, Rosendaal F R, Psaty B M. Serum lipid levels and the risk of venous thrombosis. Arterioscler Thromb Vasc Biol. 2004;24: 1970-1975.

(72) Grady D, Wenger N K, Herrington D, Khan S, Furberg C, Hunninghake D, Vittinghoff E, Hulley S. Postmenopausal hormone therapy increases risk for venous thromboembolic disease. The Heart and Estrogen/progestin Replacement Study. Ann Intern Med. 2000;132:689-696.

(73) Herrington D M, Vittinghoff E, Lin F, Fong J, Harris F, Hunninghake D, Bittner V, Schrott H G, Blumenthal R S, Levy R. Statin therapy, cardiovascular events, and total mortality in the Heart and Estrogen/Progestin Replacement Study (HERS). Circulation. 2002; 105:2962-2967.

(74) Ray J G, Mamdani M, Tsuyuki R T, Anderson D R, Yeo E L, Laupacis A. Use of statins and the subsequent development of deep vein thrombosis. Arch Intern Med. 2001;161:1405-1410.

(75) Doggen C J, Lemaitre R N, Smith N L, Heckbert S R, Psaty B M. HMG CoA reductase inhibitors and the risk of venous thrombosis among postmenopausal women. J Thromb Haemost. 2004;2:700-701.

(76) Lacut K, Oger E, Le Gal G, Couturaud F, Louis S, Leroyer C, Mottier D. Statins but not fibrates are associated with a reduced risk of venous thromboembolism: a hospital-based case-control study. Fundam Clin Pharmacol. 2004;18:477-482.

(77) Espana F, Vaya A, Mira Y, Medina P, Estelles A, Villa P, Falco C, Aznar J. Low level of circulating activated protein C is a risk factor for venous thromboembolism. Thromb Haemost 2001; 86: 1368-1373.

(78) Kaplan E L, Meier P. Nonparametric estimation from incomplete observations. J Am Stat Assoc. 1958; 53:457-481.

What is claimed is:

1. A method of determining an individual at risk for venous thrombosis comprising:
A) measuring a level of lipid or lipoprotein in a test biological specimen obtained from an individual; and
B) comparing the level of said lipid or lipoprotein in said test biological specimen to a normal range of lipid or lipoprotein for a normal biological specimen,
wherein a lower level of lipid or lipoprotein in the test biological specimen is indicative of a risk factor for venous thrombosis for the individual.

2. The method according to claim 1, wherein the biological specimen is blood, plasma, serum, cerebrospinal fluid, semen, lung fluid, lymph fluid, saliva, or urine.

3. The method according to claim 1, wherein the lipid or lipoprotein is selected from the group consisting of HDL-cholesterol, HDL particles, large HDL particles (HDL2), apolipoprotein Al, apolipoprotein ClII and apolipoprotein ClII that is not associated with apolipoprotein B.

4. A method of determining an individual's risk for venous thrombosis comprising:
A) measuring a level of lipid or lipoprotein in a test biological specimen obtained from the individual; and
B) comparing the level of said lipid or lipoprotein to a normal range of lipid or lipoprotein from a biological specimen,
wherein a higher than normal level of lipid or lipoprotein in the test biological specimen compared to a normal biological specimen is indicative of a risk factor for venous thrombosis for the individual.

5. The method according to claim 4, wherein the biological specimen is blood, plasma, serum, cerebrospinal fluid, semen, lung fluid, saliva, lymph fluid or urine.

6. The method according to claim 4, wherein the lipid or lipoprotein is selected from the group consisting of LDL-cholesterol, apolipoprotein B, and LDL particles.

7. The method according to claim 6, wherein the LDL particles are selected from the group of IDL particles and small LDL particles.

8. A method of determining an individual's risk for venous thrombosis comprising:
A) measuring the levels of at least two lipids or lipoproteins in a test biological specimen obtained from the individual;
B) determining a ratio of the level of one lipid or lipoprotein relative to the other; and
C) comparing the ratio of said lipids or lipoproteins to a normal range for the ratio of lipids or lipoproteins from a biological specimen,
wherein a higher than normal ratio of lipids or lipoproteins in the test biological specimen compared to the ratio of lipids or lipoproteins in a normal biological specimen is indicative of a risk factor for venous thrombosis for the individual.

9. The method according to claim 8, wherein the biological specimen is blood, plasma, serum, cerebrospinal fluid, semen, lung fluid, saliva, lymph fluid or urine.

10. The method according to claim 8, wherein the ratio of lipids or lipoproteins is selected from the group consisting of an apolipoprotein B/apolipoprotein Al ratio and an LDL-cholesterol/HDL-cholesterol ratio.

11. A method of determining an individual's risk for venous thrombosis comprising: determining the cholesteryl ester transfer protein (CETP) genotype of the individual, wherein a variation of the CETP genotype relative to controls is indicative of a risk factor for venous thrombosis.

12. The method according to claims 1-10 where the determination of lipid or lipoprotein or lipoprotein particle concentration is made using NMR spectroscopy.

13. The method according to claim 12, wherein the lipid or lipoprotein is selected from the group consisting of LDL, LDL-cholesterol, LDL small particles, IDL particles, HDL-cholesterol, HDL particles and large HDL particles.

14. A method of reducing an individual's risk for venous thrombosis comprising administration of a lipid-altering drug in an amount sufficient to reduce the individual's risk for venous thrombosis.

15. The method according to claim 14, wherein the lipid-altering drug is selected from the group consisting of statins, CETP inhibitors, nicotinic acid, fibrates, bile acid sequestrants, and drugs that elevate HDL-cholesterol or large HDL particles and/or that lower LDL-cholesterol or small LDL particles.

16. The method according to claim 11, comprising: determining the presence of the B1 allele of the CETP TaqI polymorphism wherein the presence of this genotype in the test biological specimen is indicative of a risk factor for venous thrombosis for the individual.

17. The method according to claim 11, comprising: determining the presence of CETP gene variants coding for Pro373 and/or for Gln451, wherein the presence of either one or both gene variants in the test biological specimen is indicative of a risk factor for venous thrombosis for the individual.

18. A method of determining an individual at risk for venous thrombosis comprising: A) measuring a level of CETP mass or activity in a test biological specimen obtained from an individual; B) comparing the level of said CETP mass or activity in said test biological specimen to a normal range of CETP mass or activity for a normal biological specimen wherein a higher level of CETP in the test biological specimen is indicative of a risk factor for venous thrombosis for the individual.
